(19) United States
(12) Patent Application Publication
(10) Pub. No.: US 2022/0073874 A1
(43) Pub. Date: Mar. 10, 2022

(54) METHODS FOR ENHANCING DIRECT REPROGRAMMING OF CELLS

(71) Applicant: UNIVERSITY OF SOUTHERN CALIFORNIA, Los Angeles, CA (US)

(72) Inventors: Justin Ichida, Los Angeles, CA (US); Kimberley N. Babos, Los Angeles, CA (US); Kate E. Galloway, Los Angeles, CA (US)

(21) Appl. No.: 17/472,424

(22) Filed: Sep. 10, 2021

Related U.S. Application Data
(60) Provisional application No. 63/076,931, filed on Sep. 10, 2020.

(51) Int. Cl. C12N 5/0793; C12N 5/074

(52) U.S. Cl. CPC C12N 5/0619; C12N 5/0696; C12N 2501/48; C12N 2501/20; C12N 2501/15

(57) ABSTRACT

The disclosure relates methods for increasing the efficiency of cellular reprogramming of somatic cells and improving the maturity of the resulting cells.

### CROSS REFERENCE TO RELATED APPLICATIONS

This application claims priority to U.S. Provisional Application No. 63/076,931, filed Sep. 10, 2020, the disclosure of which is incorporated herein by reference.

### STATEMENT OF GOVERNMENT SUPPORT

This invention was made with government support under Grant No. 1R01NS097850-01 awarded by the National Institutes of Health (NIH). The government has certain rights in the invention.

### FIELD OF THE INVENTION

The invention relates methods for increasing the efficiency of cellular reprogramming of somatic cells and improving the maturity of the resulting cells.

### BACKGROUND

Cellular reprogramming redirects the transcriptional state of a cell to a new fate. By supplying inaccessible somatic cell types in unique genomic contexts, transcription factor-mediated reprogramming massively expands the potential for in vitro disease modeling. However, epigenetic barriers limit reprogramming between somatic lineages to rare events and cause incomplete conversion of gene regulatory networks (GRNs). Efforts to identify epigenetic factors limiting reprogramming have focused primarily on induced pluripotent stem cell (iPSC) generation, and many of these findings are specific to iPSC reprogramming.

### SUMMARY

Although cellular reprogramming enables the generation of new cell types for disease modeling and regenerative therapies, reprogramming remains a rare cellular event. By examining reprogramming of fibroblasts into motor neurons and multiple other somatic lineages, epigenetic barriers to conversion can be overcome by endowing cells with the ability to mitigate an inherent antagonism between transcription and DNA replication. The disclosure shows that transcription factor overexpression induces unusually high rates of transcription and that sustaining hypertranscription and transgene expression in hyperproliferative cells early in reprogramming is critical for successful lineage conversion. However, hypertranscription impedes DNA replication and cell proliferation, processes that facilitate reprogramming. The disclosure provides a chemical and genetic cocktail that dramatically increases the number of cells capable of simultaneous hypertranscription and hyperproliferation by activating topoisomerases. Further, the disclosure provides that hypertranscribing, hyperproliferating cells reprogram at 100-fold higher, near deterministic rates. Therefore, relaxing biophysical constraints overcomes molecular barriers to cellular reprogramming.

In a particular embodiment, the disclosure provides a method for increasing the efficiency of cellular reprogramming and improving the maturity of the resulting cells by form a population of hypertranscribing, hyperproliferating cells (HHCs), comprising: contacting a population of cells with a cocktail that comprises a TGF-β inhibitor, and a dominant negative p53 mutant, to form a population of HHCs; and isolating the population of HHCs. In a further embodiment, the TGF-β inhibitor is selected from RepSox, SB431542, A-83-01, LY-364947, LY2157299, LY-364947, A 83-01, and ALK5 inhibitor. In a certain embodiment, the TGF-β inhibitor is RepSox. In another embodiment, the dominant negative p53 mutant lacks a DNA-binding domain. In yet another embodiment, the dominant negative p53 mutant is p53DD. In a further embodiment, the cocktail further comprises a Ras mutant. In yet a further embodiment, the Ras mutant is hRAS G12V. In another embodiment, the cocktail relieves DNA supercoiling by activating topoisomerases. In yet another embodiment, the cells are stem cells. In a further embodiment, the stem cells are embryonic stem cells or induced stem cells. In yet a further embodiment, the cells are induced motor neuronal cells (iMNs). In another embodiment, the iMNs are derived from fibroblasts. In yet another embodiment, the population of HHCs is converted into neurons. In a further embodiment, the neurons are characterized as being electrophysiology mature.

### BRIEF DESCRIPTION OF THE DRAWINGS

FIG. 1A-L shows Genetic and Chemical Factors Relieve Genomic Stress and Reprogramming Block. (A) Binucleated iMN at 14 dpi. Scale bar represents 10 μm. (B) Mitotic cell with a micronucleus at 2 dpi. Arrow denotes micronucleus. Scale bar represents 5 μm. Mitotic cells were identified based on morphology of DAPI+ nuclei. (C) Percentage of mitotic anaphase-telophase cells with a micronucleus at 2 dpi. Anaphase-telophase cells with a non-integrated DNA fragment were scored as having micronuclei. n=150-175 cells from 3-6 independent conversions per condition. Percentage±95% confidence interval is shown; chi-square test. (D) Mitotic cell with a chromatin bridge at 4 dpi. Arrow denotes bridge. Scale bar represents 10 μm. (E) Percent of mitotic anaphase-telophase cells with a chromatin bridge at 4 dpi. Anaphase-telophase cells with a DNA strand between daughter cells were scored as having a bridge. n=63-100 cells from 3-6 independent conversions per condition. Percentage±95% confidence interval per condition is shown; chi-square test. (F) Legend of genetic and chemical combinations used in conversion. 6F, 6 transcription factors only; 6FDD, 6 transcription factors and p53DD, a p53 mutant; 6FDDRR, 6 transcription factors and p53DD, hRasG12V, and RepSox. (G) SF iMNs, 14 dpi. Scale bar represents 100 μm. (H) SFDDRR-iMNs, 14 dpi. Scale bar represents 100 μm. (I) iMN yield in 6F, 6FDD, or 6FDDRR conditions at 14 dpi. Conversion yield determined by counting Hb9::GFP+ cells with neuronal morphology divided by number of cells seeded is shown. n=10-20 independent conversions per condition. Mean±SEM; one-way ANOVA. (J) Percentage of mitotic anaphase-telophase cells with a micronucleus at 2 dpi. n=100 cells from 3 independent conversions per condition. Percentage±95% confidence interval is shown; chi-square test. (K) Percentage of mitotic anaphase-telophase cells with a chromatin bridge at 4 dpi. n=100 cells from 3 independent conversions per condition. Percentage±95% confidence interval is shown; chi-square test. (L) Percentage of binucleated iMNs at 14 dpi; n=6 independent conversions; mean±SEM; unpaired t test. Significance summary: p>0.05 (ns); *p≤0.05; **p≤0.01; ***p≤0.001; and ****p≤0.0001.

FIG. 2A-R shows Hypertranscribing and Hyperproliferating Cells Drive Reprogramming. (A) FAGS plot showing relative EU incorporation in viable cells from SF-infected MEF cultures at 1 and 2 dpi. Cell viability was determined by forward scatter (FSC) and side scatter (SSC) profiles in FAGS analysis. (B) Relative transcription rate measured by EU incorporation via flow cytometry at 1 and 2 dpi in SF-infected MEFs compared to uninfected control. Mean EU intensity of non-transduced MEFs=1. Only viable cells, determined by FSC and SSC profile via FAGS, were analyzed. n=5 independent transductions per condition. Mean±SEM; unpaired t test. (C) Schematic of CSFE-based flow sorting and replating of populations for reprogramming assays. (D) CFSE intensity measured by flow cytometry at 4 dpi. “HyperP,” hyperproliferating cells, defined as cells showing a two-fold increase in division rate (an 8-fold decrease in CFSE intensity) compared to the average of Controi-Puro MEFs. (E) CFSE intensity measured by flow cytometry for Puro-infected cells (control), Ascl1-infected cells, or Bm2+Ascl1+Mytl1-infected cells (BAM) at 4 dpi. (F) Effect of addition of Ascl1 or neuronal reprogramming factors BAM on the percentage of hyperproliferating cells measured by flow cytometry at 4 dpi. n=3-6 independent transductions per condition. Mean±SEM; one-way ANOVA. (G) CFSE intensity measured by flow cytometry at 4 dpi with gates showing CFSE-Iow (HyperP) and CFSE-high. (H) Yield of iMNs from reprogramming populations sorted by CFSE intensity (CFSE-Iow and CFSE-high) at 4 dpi. Percent yield determined by counting total iMNs normalized by total number of cells counted per population at 4 dpi is shown. n=4-23 independent conversions per condition. For 6F and 6FDD, median±lnterquartlle range and Mann-Whitney test between CFSE high and low groups In each transduction condition are shown. For 6FDDRR, mean±SEM. Unpaired t test between CFSE high and low groups is shown. (I) Schematic of CFSE-EU assay for measuring transcription and proliferation rates via flow cytometry at 4 dpi. (J) Dot plot of CFSE intensity and fluorescently labeled EU for Control-Puro (gray), 6F (green), and 6FDDRR (red). Histograms of CFSE and EU intensity adjacent to dot plot are shown. Quadrant to demark HHCs sat by reference to 6F condition is shown. Hyparprollferatlng and slow cycling calls sat by selecting CFSE value In SF condition to allow the dimmest 15% are shown. High EU values set by top half of SF condition are shown, resulting in ˜7% HHCs in SF. (K) Relative transcription rate measured by EU incorporation via flow cytometry at 4 dpi of the whole population (all calls) of 6F-infected cells compared to hyperprollferativa cells measured in 6F-Infacted MEFs. n=10 Independent transductions per condition. Mean±SEM; one-way ANOVA (U Percent relative transcription rate increase upon inhibition of DNA synthesis with aphidicolin treatment at 4 dpi. (L) Relative transcription rate determined by difference between rates with and without aphidicolin treatment normalized to without for each transduction condition. N=3 independent transductions per condition. Mean±SEM; unpaired t test between with and without aphidicolin treatment for each transduction condition. (M) Percentage of HHCs. n=11-16 Independent conversions per condition. Median±lnterquartlle range Is shown; Kruskai-Wallls test. (N) Yield of Hb9::GFP+ cells counted via flow cytometry at 17 dpi normalized to number of seeded cells. n=7-8 independent conversions per condition. Mean±SEM; unpaired t test. (O) Yield of Hb9::GFP+ cells normalized to total cell number at 17 dpi. Calls ware quantified via flow cytometry at 17 dpi. n=7 or 8 independent conversions per condition. Mean±SEM; unpaired t test. (P) Schematic of CFSE-EU-pulselaba111888y to sort and label HHCs at 4 dpi followed by evaluation of Hb9::GFP intensity at 8 dpi. (Q) Percentage of Hb9::GFP+ cells in 6FDDDR conditions for various gated populations. Cells gated for low EU intensity are shown. EU-low, cells with EU intensity in the lowest three quartiles, and EU-high, cells with EU intensity in the top quartile, at 8 dpi compared to all viable cells are shown, both EU-high and EU-Iow (all cells). VIable calls deHned basad on FSC and sse profiles via FACS are shown. n=7 or 8 Independent conversions. Meen±SEM; ona-way ANOVA. (R) Percentage of replated hyperproliferating cells in 6FDDRR conditions gated for high EU intensity (cells with EU intensity in the top quartile as measured by FACS) at 8 dpi. By definition, the whole viable population (alij contained 25% EU-Hi cells and Hb9::GFP+ and Hb9::GFP+ Bright calls (Hb9::GFP intensity in the top half of all viable Hb9+ cells) displayed enrichment of EU-hlgh cells. n=4-8 Independent conversions. Median±lnterquartlle range is shown; KruskaiWallistest. Significance summary: p>0.05 (ns); *p≤0.05; **p≤0.01; . . . ***p≤0.001; and ****p≤0.0001.

FIG. 3A-H shows Sustained Transgene Expression Differentiates Complete from Partial Reprogramming. (A) Hb9::GFP+ cells with fibroblast (top) or neuronal (bottom) morphology at 17 dpi. Scale bars represent 20 μm. (B) Percentage of Hb9::GFP+ cells of all viable cells measured by flow cytometry at 8 dpi. n=6 independent conversions per condition. Mean±SEM; onewayANOVA. (C) Percentage of Hb9::GFP+ cells with neuronal morphology of total Hb9::GFP+ cells at 17 dpi. n=9 independent conversions per condition. Mean±SEM; onewayANOVA. (D) Relative gene expression of cells collected at 14 dpi sorted based on No, Low, or Bright Hb9::GFP expression. Bright Hb9::GFP, cells in the top 50% ofHb9::GFP in the 6F condition. Gene expression was calculated based on qRT-PCR data. The expression level that was highest among the three conditions was set to 1 and used to normalize levels for the other two conditions. n=2 independent experiments for each condition. (E) Relative expression for single cells with either fibroblast (n=16) or neuronal (n=39) morphology for qPCR assays for endogenous Ngn2 and/s/1 and viral/s/1 (vlsl1). Median±interquartile range is shown; Mann-Whitney test. (F) Relative lsi1-GFP intensity in all viable cells (all) and HyperP infected with lsi1-GFP and 6F or 6FDDRR measured by flow cytometry at 4 dpi. n=4-6 independent transductions per condition. Mean±SEM; one-way ANOVA. (G) Percentage of lsi1-GFP+ cells in all viable cells (aiO and HyperP measured by flow cytometry at 4 dpi. lsi1-GFP+ determined by expression exceeding fluorescein isothiocyanate (FITC) values for untransfected cells is shown. n=6 independent transductions per condition. Mean±SEM; one-way ANOVA. (H) Relative integrations of lsi1-GFP and NIL viruses in cells collected at 4 dpi. Relative integrations determined by qPCR are shown. Delta Ct of transgene calculated by difference of Ct between transgene and endogenous genomic region is shown. Relative integrations calculated by normalizing to NIL condition are shown. n=3 independent transductions per condition. Mean±SEM; one-way ANOVA. Significance summary: p>0.05 (ns); *p::′>0.05; **p::′>0.01; ***p::′>0.001; and ****p::′>0.0001.

FIG. 4A-O shows Topoisomerase Expression Enables Cells to Exhibit Both Hyperproliferation and Hypertranscription. (A) Schematic of populations collected across conversion and profiled via single-cell RNA-seq. Individual libraries were prepared for MEFs (1,357 cells), hyperproliferating cells (CFSE-low) for 6F (1,174 cells) and 6FDDRR (1,189 cells) collected at 4 dpi (6F 4 dpi and 6FDDRR 4 dpi), and Hb9::GFP+ cells for 6F (259 cells) and 6FDDRR (406 cells) at 8 dpi (6F 8 dpi and 6FDDRR 8 dpi) and 6F iMNs (1,863 cells) and 6FDDRR iMNs (2,869 cells) at 14 dpi (iMNs). (B) t-distributed stochastic neighbor embedding (tSNE) projection of all cells mapped during reprogramming colored by condition. (C) Distribution of pseudotime across cells in each condition. (D) Relative UMI distribution across cells. (E) Clustering of three cellular states across the tSNE projection. (F) Relative expression of Col1a1, Mki67, Top2a, Top1, and Map2 over pseudotime. Colors correspond to states identified in (E). {G) Violin plot of UMI (top, unique molecular identifiers) and relative Mki67 expression (bottom) for clusters identified in (E). (H) Violin plot of relative expression of Top1 (top) and Top2a (bottom) for clusters identified in (E). (I) Reads from Top1 and Top2a quantified by cell number normalized (CNN) RNA-seq at 4 dpi. (J) Percentage of mitotic anaphase-telophase cells with a chromatin bridge at 4 dpi for 6FDDRR conditions. n=3 independent conversions per condition, n=SQ—70 cells per condition. Percentage±95% confidence interval; chi-square test. (K) Percentage of HHCs in 6FDDRR conditions. n=4-6 independent conversions per condition. Mean±SEM; one-way ANOVA. (L) Percentage of HHCs in 6FDDRR conditions treated for 18 h with camptothecin (Cpt) or doxorubicin (Doxo) prior to 4 dpi compared to DMSO control. n=4 or 5 independent conversions per condition. Mean:1: SEM; one-way ANOVA. (M) Yield of IMNs In 6DDDRR conditions at 14 dpl. n=7-9 Independent conversions per condition. Mean±SEM; one-way ANOVA. (N) Yield of iMNs at 14 dpi in 6FDDRR conditions treated for 18 h with Cpt or Doxo prior to 4 dpi compared to DMSO control. n=3 or 4 independent conversions par condition. Mean±SEM; one-way MOVA. (O) Yield of IMNs at 14 dpl In 6FDD condition treated with RepSox with or without Top1 overexpression. n=7-9 Independent conversions par condition. Mean:1: SEM; unpaired t test. Significance summary: p>0.05 (ns); •p≤0.05; P≤0.01; P≤0.001: and P≤0.0001.

FIG. 5A-P shows DDRR and Topoisomerase Expression Reduces Negative DNA Supercoiling and R-Loop Formation and Sustains Transcription in S-Phase (A) Psoralen incorporation at 4 dpi. Scale bars represent 10 μm. Dotted white lines outline the nucleus. (B) Mean intensity of biotinylated psoralen conjugated streptavidin-Aiexa Fluor 594 at 4 dpi. Cultures treated with 1 μM aphidicolin for 2 h prior to collection at 4 dpi are shown. n=42-130 cells from 3 independent conversions per condition. Median±interquartile range is shown; Kruskai-Wallis test. (C) Mean intensity of biotinylated psoralen conjugated streptavidin-Aiexa Fluor 594 at 4 dpi in 6FDDRR conditions at 4 dpi. n=99-162 cells from 3 independent conversions per condition. Median±interquartile range is shown; Kruskai-Wallis test. (D) Relative amount of DNA protected by exonuclease digestion in regions 500 bp upstream of transcription start sites for listed genes at 4 dpi. n=4 independent transductions per condition per gene. Mean±SEM; unpaired t test. (E) R-loop immunostaining (S9.6) at 4 dpl. Scale bars represent 10 μM. Dotted white lines outline the nucleus. (F) A-loop intensity per area at 4 dpi. n=101-158 cells from 3 independent conversions per condition. Median±interquertile range is shown; Kruskai-Wallis test. (G) A-loop intensity per area at 4 dpi in 8FDDRR conditions. n=119-135 cells from 3 independent conversions per condition. Median±interquartile range is shown; Kruskai-Wallis test. (H) DNA fiber labeling scheme to Identify progressing replication forks (red-green), stalled forks (red only), and new or1glns (green only). (I) Relative number of stalled replication forks at 4 dpi. Stalled replication forks were quantified and normalized to all replicative fiber species to generate the percentage of stalled replication forks n=1,000 fibers per condition from 4 independent transductions. Percentage±95% confidence interval is shown; Fisher's exact test. (J) Relative number of new origins at 4 dpi. New origins were quantified and normalized to all replicative fiber species to generate the percentage of stalled replication forks. n=1,000 fibers per condition from 4 independent transductions. Percentage±95% confidence interval is shown; Fisher's exact test. (K) Dot plot of EdU and active RNA polymerase II intensity at 4 dpi for Control-Pure (gray), 6F (green), and 6FDDRR (red). Gating to demark S phase cells with high active RNAPII (RNAPII Ser2p) Is shown. S phase determined by Intensity above EdU Incorporation In non-proliferative, irradiated MEFs Is shown (Rgure S4H). High RNAPIISer2p, the top quartile of RNAPIISer2p intensity in Control-Pure infected cells in S phase cells. (L) Percentage of cells in S phase with high RNAPII activity from area gated in (K) measured via flow cytometry at 4 dpi. Percentage relative to total viable cell population based on FSC and SSC profile via FACS is shown. n=4 independent conversions per condition. Mean±SEM; one-way MOVA. (M) Relative DNA synthesis rate of S phase cells at 4 dpl. Relative DNA synthesis rate determined by EdU Intensity of S phase population normalized to EdU intensity of S phase population in Control-Pure condition is shown. n=4 independent conversions per condition. Mean±SEM; one-way ANOVA. (N) Relative active RNAPII of S phase cells at 4 dpi. Relative active RNAPII rate in S phase cells determined by intensity of RNAPII Ser2p in S phase population normalized to intensity of RNAPII Ser2p in S phase population in Control-Pure condition is shown. n=4 independent conversions per condition. Mean±SEM; one-way MOVA. (O) Dot plot of EdU and active RNA polymerase II intensity at 4 dpi for 8FDDRR+Scrambled shRNA (gray), 8FDDRR+ahTop2a (blue), and 8FDDRR+shTop1 (red) shRNAs. Gating to demark S phase cells with high active RNAPII (RNAPII Ser2p) is shown. (P) Percentage of cells In S phase with high RNAPII activity from area gated In (O) at 4 dpi in 6FDDRR conditions. n=4 Independent conversions per condition. Mean±SEM; one-way ANOVA. Significance summary: p>0.05 (ns); •p≤0.05; P≤0.01; P≤0.001; and P≤0.0001.

FIG. 6A-M shows that converting HHCs Adopt the Induced Motor Neuron Transcriptional Program and Accelerate Morphological Maturation. (A) RNA-seq heatmap for Hb9::GFP+ cells at 17 dpi from different conditions compared to starting MEFs across the 1,186 genes that are differentially expressed between MEFs and Hb9::GFP+ cells. n=3 independent conversions per condition. (B) Volcano plot comparison of genes up- (blue) or downregulated (red) in Hb9::GFP+ cells at 17 dpi. (C) List of gene ontology (GO) terms for genes upregulated (top, blue) or downregulated (bottom, red) in 6FDDRR cells compared to 6F at 17 dpi. (D) tSNE projection of Hb9::GFP+ embryonic motor neurons (embMNs) collected at 12.5 dpi and iMNs generated by three different cocktails (SF, SFDDRR, and 6FDDRR+Top1) colored by individual condition. embMNs were bioinformatically identified by/s/1 expression to distinguish from other Hb9::GFP+ populations. (E) Relative expression colored by intensity of Co/la1, /s/1, Map2, and Chat over the populations in the tSNE in (D). (F) Hb9::GFP+ iMNs immunostained for Map2 at 17 dpi. Scale bars represent 5 μm. (G) Percentage of the Hb9::GFP+ cell population with neuronal gene expression profile at 17 dpi. (H) Relative expression of neurosignaling genes (i.e., Scg2, Chgb, Sncg, and Snca) colored by intensity over the populations in the tSNE in (D). (I) List of gene ontology (GO) terms for marker genes upregulated in iMN clusters. (J) Percentage of multipolar iMNs derived from MEFs at 14 dpi. n=6 or 7 independent conversions per condition. Mean±SEM; unpaired t test. (K) SFA ratio evoked action potentials (Aps) of mouse iMNs at 14 dpi. n=7 or 8 cells from 3 independent conversions per condition. Median±interquartile range is shown; Mann-Whitney test. (L and M) Representative action potentials evoked in mouse iMNs by a positive current injection (indicated by solid bar across bottom) illustrating SFA over the course of the stimulus of iMNs in 6FDD (M) and 6F (L) conditions at 14 dpi. Significance summary: p>0.05 (ns); *p≤0.05; **p≤0.01; ***p≤0.001; and ****p≤0.0001.

FIG. 7A-M shows the DDRR Cocktail Boosts Reprogramming across Multiple Cell Types and Species. (A) Yield of induced neurons for different conditions, including control with 3 factors (3F [Bm2, Asc/1, and Mytl m, 3FDD, and 3FDDRR counted by MAP2+ cells at 17 dpi over number of cells seeded. n=6 or 7 independent conversions per condition. Mean±SEM; one-way ANOVA. (B) Yield of induced dopaminergic neurons (iDANs) for different conditions, including control with 5 factors (5F [8m2, Asc/1, Mytl1, Lmx1A, and FoxA2]), 5FDD, and 5FDDRR counted by MAP2+ cells at 17 dpi. n=6-8 independent conversions per condition. Mean±SEM; one-way ANOVA. (C) Yield of induced inner ear hair cells (iHCs) for different conditions, including control with 3 factors (3F [Bm3C, Afoh1, and Gfi1]), 3FDD, and 3FDDRR counted by Atoh1::nGFP+ cells at 17 dpi. n=3-16 independent conversions per condition. Mean±SEM; one-way ANOVA. (D) 3F-iHCs and 3FDD-iHCs immunostained with Myosin VIla at 17 dpi. Scale bars represent 100 μm. (E) Yield of iMNs generated from adult tail tip fibroblasts with 6F, 6FDD (both conditions with RepSox), and 6FDDRR at 28 dpi. n=4-9 independent conversions per condition. Mean±SEM; one-way ANOVA. (F) Yield of iMNs generated from Hb9::GFP+ adult mouse muscle explants at 28 dpi. n=4 or 5 independent conversions per condition. Mean±SEM; unpaired t test. (G) Yield of iMNs generated from human fibroblasts with factors alone (7F) or 7FDD (both conditions with RepSox), counted by MAP2+ cells at 35 dpi. n=4-6 Independent conversions per condition. Medlen±lnterquartlle range is shown; Mann-Whitney test. (H) Percentage of multipolar iMNs derived from primary human fibroblasts at 35 dpi. n=3 independent conversions per condition. Mean±SEM; unpaired t test. {I and J) Step voltage depolarizations result in functional sodium and potassium channels in human 7F (I) or 7FDD-iMNs (J) at 35 dpi. (K and L) Action potentials evoked by step current injection In current-clamp configuration for human 7F (K) or 7FDD-IMNs at 35 dpl (L). (M) Model of topoisomerase-mediated reprogramming through hypertranscribing, hyperproliferating cells. Significance summary: p>0.05 (ns); •p≤0.05; **P≤0.01; ***P≤0.001; and ****P≤0.0001.

FIG. 8A-G shows genetic and chemical factors relieve genomic stress and reprogramming block. (A) Time series of DsRed-MEFs infected with 6F or 6FDD from 69 to 124 hours post-infection. Division event observed at 72 hours, Hb9::GFP activation observed at 104 hours. Arrow(s) indicate cell that divides prior to activation of Hb9::GFP in the daughter cells. (B) Percentage of mitotic anaphase-telophase cells with chromatin bridges at 4 dpi for uninfected control (UIC), Control-Puro infected cells, and 6F conditions. Anaphase-telophase cells with one or more DNA strands between the separating/separated daughter cells were determined as having a bridge. n=63-100 cells from 3-6 independent conversions per condition. Significance determined using a Chi-square test to compare the frequency in encountering a mitotic cell with a chromatin bridge between conditions. Percentage+/−95% confidence interval. (C) mRNA levels of Mbd3 in 6F conditions treated with two Mbd3 shRNAs at day 0 at collected at 2 dpi. mRNA levels are shown relative to a scrambled shRNA control. All Mbd3 mRNA levels are significantly lower (p<0.05) in the shRNA conditions than in the scrambled controls. n=5-6 independent transductions per condition. Mean+/−s.e.m. Unpaired t-test. (D) Yield of iMNs for 6F conditions in presence of scrambled or Mbd3 shRNAs at 14 dpi. n=8-23 independent conversions per condition. Median+/−interquartile range. Kruskal-Wallis test. (E) Yield of iMNs for 6F condition in presence of scrambled or titration of Mbd3-A shRNA at 14 dpi. n=4-5 independent conversions per condition. Mean+/−s.e.m. One-way ANOVA. (F) mRNA levels of Gatad2a in 6F cultures treated with two Gatad2a shRNAs at day 0 and collected at 2 dpi. mRNA levels are shown relative to a scrambled shRNA control. All Gatad2a mRNA levels are significantly lower (p<0.05) in the shRNA conditions than in the scrambled controls. n=4 independent transductions per condition. Mean+/−s.e.m. Unpaired t-test. (G) Yield of iMNs for 6F condition in presence of scrambled or Gatad2a shRNAs at 14 dpi. n=9-16 independent conversions per condition. Median+/−interquartile range. Kruskal-Wallis test. Significance summary: p>0.05 (ns), *p≤0.05, **p≤0.01, ***p≤0.001, ****p≤0.0001.

FIG. 9A-T shows Hypertranscribing and hyperproliferating cells drive reprogramming. (A) Representative image of EU-click labeling and nucleolin immunostaining in 6F MEFs at 1 and 2 dpi. Scale bars represent 10 μm. (B) Mean EU intensity within or excluding nucleoli in 6F MEFs at 1 and 2 dpi. n=56 cells (nucleolar EU) or 57 cells (non-nucleolar EU) from 3 independent conversions per condition. Median+/−interquartile range. Mann-Whitney test between 1 and 2 dpi samples within each nuclear compartment. (C) Percentage of Ki67+ cells in Control-Puro, 6F, or 6FDDRR conditions measured via flow cytometry at 4 dpi. n=3-4 independent transductions per condition. Mean+/−s.e.m. One-way ANOVA. (D) Representative histogram of CFSE intensity measured by flow cytometry for MEFs infected with DsRed or 6F at 4 dpi. (E) Percentage of hyperproliferating cells for MEFs passage 1-6 measured via CFSE using flow cytometry. Hyperproliferating cells were defined as cells showing a two-fold increase in division rate (i.e. an eight-fold decrease in CFSE intensity) compared to the average of the control population, which was comprised of passage 1 MEFs. n=3-5 biological replicates per condition. Mean+/−s.e.m. One-way ANOVA. (F) Effect of MEF passage on iMN yield for 6F condition. n=3-4 independent conversions per condition. Mean+/−s.e.m. One-way ANOVA. (G) Schematic of timeline for mitomycin C treatment and effect on conversion yield for the 6FDD condition at 14 dpi. n=3 independent conversions per condition. Mean+/−s.e.m. One-way ANOVA. (H) Conversion yield in 6F and 6FDD conditions in absence or presence of p21 overexpression at 14 dpi. n=5-7 independent conversions per condition. Mean+/−s.e.m. Unpaired t-test of dsRed vs p21 within each reprogramming cocktail. (I) Percentage of cleaved caspase-3+ cells at 2, 4, and 8 dpi in Control-Puro, 6F, or 6FDDRR conditions measured via flow cytometry. n=3-5 independent transductions per condition. Mean+/−s.e.m. One-way ANOVA between conditions at each day. (J) Scatter plot showing effect of 18-hour 1 M aphidicolin treatment on cells as measured by EdU incorporation and DAPI via flow cytometry at 4 dpi. (K) Percentage of cells in S-phase in 6FDDRR condition with DMSO or Aphidicolin treatment for 18 hrs and measured with EdU incorporation via flow cytometry at 4 dpi. n=4 independent transductions per condition. Mean+/−s.e.m. Unpaired t-test. (L) Effect of aphidicolin treatment on total number of viable cells in 6FDDRR condition at 4 dpi. Viable cells were defined based on their FSC and SSC profile via FACS. n=5-6 independent transductions per condition. Mean+/−s.e.m. Unpaired t-test. (M) Percent viable cells in 6FDDRR condition at 4 dpi following 18-hrs treatment with water or -Amanitin. Viable cells were defined based on their forward scatter (FSC) and side scatter (SSC) profile via FACS. n=6 independent transductions per condition. Mean+/−s.e.m. Unpaired t-test. (N) Relative transcription rate following 18-hour α-Amanitin treatment in 6FDDRR conditions as measured by EU incorporation via flow cytometry at 4 dpi. The mean EU intensity of 6FDDRR cells treated with water was defined as a relative transcription rate of 1. n=6 independent transductions per condition. Mean+/−s.e.m. Unpaired t-test. (O) Effect of α-Amanitin treatment on the yield of iMNs in 6FDDRR condition at 14 dpi. n=8 independent conversions per condition. Mean+/−s.e.m. Unpaired ttest. (P) Yield of iMNs in 6F condition with TBP overexpression at 14 dpi. n=8-21 independent conversions per condition. Mean+/−s.e.m. Unpaired t-test. (Q) CFSE intensity of cells treated with CFSE at 1 dpi and flow sorted at 4, 6, and 8 dpi in 6FDDRR conditions. n=3 independent transductions per condition. Mean+/−s.e.m. One-way ANOVA. AU=arbitrary units. (R) EU intensity in cells pulsed with EU for 4 hours at 4 dpi and fixed at 4, 6, and 8 dpi in 6FDDRR condition. EU intensity measured by flow cytometry. n=3 independent transductions per condition. Mean+/−s.e.m. One-way ANOVA. AU=arbitrary units. (S) GFP intensity of Hb9::GFP+ cells at 14 dpi correlates with neuronal morphology, increasing from fibroblast to neuronal cells (left to right). Scale bar represents 100 μm. (T) Side-by-side comparison of dim fibroblast-like and bright neuronal Hb9::GFP+ cells at 14 dpi Significance summary: p>0.05 (ns), *p≤0.05, **p≤0.01, ***p≤0.001, and ****p≤0.0001.

FIG. 10A-K shows Sustained transgene expression differentiates complete from partial reprogramming. (A) Time series of Hb9::GFP+ intermediate to iMN. (B) Converting iMNs in the 6F condition at 10 dpi with mixed morphologies. Scale bar represents 100 μm. Arrows denote fibroblast-like cells. (C) Longitudinal tracking of cells to measure the rate at which Hb9::GFP+ intermediates adopt neuronal morphology. n=65-80 cells in the 6F condition and n=1200-1400 cells tracked in the 6FDD condition. (D) Relative expression of viral Isl1 (vIsl1), viral Lhx3 (vLhx3), viral Ngn2 (vNgn2), or endogenous Isl1 of cells collected at 14 dpi sorted based on no, low, or bright Hb9::GFP expression. n=3 independent transductions per condition. Mean+/−s.e.m. One-way ANOVA within each dpi. (E) Heatmap of relative expression for single cells with either fibroblast (top gray, n=16) or neuronal (top green, n=39) morphology for qPCR assays for fibroblast (side gray) or neuronal (side green) genes. Cells were picked at 14 dpi. (F) Relative expression for single cells with either fibroblast (n=16) or neuronal (n=39) morphology for qPCR assays for endogenous Lhx3 for cells picked at 14 dpi. Median+/−interquartile range. Unpaired t-test. (G) Yield of iMNs in 6F conditions or with 5F (i.e. Ascl1, Brn2, Mytl1, Ngn2, Lhx3)+Isl1-GFP at 14 dpi. n=6-7 independent conversions per condition. Mean+/−s.e.m. Unpaired t-test. (H) Percentage of 6F or 6FDDRR cells expressing a fluorescent protein. Cells were infected with a single fluorescent protein (e.g. individual viruses YFP or RFP) and measured via flow cytometry at 4 dpi. n=3 independent transductions per condition. Mean+/−s.e.m. One-way ANOVA. (I) Percentage of 6F or 6FDDRR cells infected with both YFP and RFP and expressing either fluorescent protein alone or both. Cells were measured via flow cytometry at 4 dpi. n=3 independent transductions per condition. Mean+/−s.e.m. One-way ANOVA. (J) Percent HHCs measured at 4 dpi with the multicistronic NIL virus (NIL) and NIL+DDRR. Hyperproliferating cells were defined as cells showing a two-fold increase in division rate (i.e. an eight-fold decrease in CFSE intensity) compared to the average of the control population, which was comprised of NIL-infected MEFs. n=3 independent transductions per condition. Mean+/−s.e.m. Unpaired t-test. (K) Yield of iMNs in NIL and NIL+DDRR conditions at 14 dpi. n=4-5 independent conversions per condition. Mean+/−s.e.m. Unpaired ttest. Significance summary: p>0.05 (ns), *p≤0.05, **p≤0.01, ***p≤0.001, ****p≤0.0001.

FIG. 11A-I shows Topoisomerase expression enables simultaneous hypertranscription, hyperproliferation in HHCs. (A) mRNA levels of Top1 and Top2a in 6FDDRR cultures treated with Top1 or Top2a shRNAs at day 0 and collected at 2 dpi. mRNA levels are shown relative to a scrambled shRNA control. All Top1 or Top2a mRNA levels are significantly lower (p≤0.05) in the Top1 or Top2a shRNA conditions than in the scrambled controls. n=5 independent transductions per condition. Mean+/−s.e.m. Unpaired t-test for each shRNA vs the scrambled control. (B) Percentage of mitotic anaphase-telophase cells with a chromatin bridge at 4 dpi for 6FDDRR conditions treated with Scrambled, Top1-B, or Top2a-B shRNAs. Anaphase-telophase cells with one or more DNA strands between the separating/separated daughter cells were determined as having a bridge. n=55-75 cells from 3 independent conversions per condition. Significance determined using a Chi-square test to compare the frequency in encountering a mitotic cell with a chromatin bridge between conditions. Percentage+/−95% confidence interval. (C) Effect of second Top1 or Top2a shRNAs on percentage of HHCs in 6FDDRR condition measured at 4 dpi via CFSE-EU flow cytometry assay as previously described. n=2-5 independent transductions per condition. Mean+/−s.e.m. for scrambled and Top1-B. Mean+/−standard deviation for Top2a-B. Unpaired t-test between scrambled and Top1-B. (D) Scatter plots showing EdU incorporation and DAPI via flow cytometry at 4 dpi in 6FDDRR+DMSO or 0.25 uM Doxorubicin for 18 hr and irradiated feeders. (E) Effect of 18-hour DMSO, camptothecin, or doxorubicin treatment on relative viability of all cells sorted in 6FDDRR condition at 4 dpi measured via flow cytometry. Relative viability determined based on FSC and SSC profiles via FACS. n=3-15 independent transductions per condition. Mean+/−s.e.m. One-way ANOVA. (F) Effect of second Top1 or Top2a shRNAs on conversion yield of iMNs in 6FDDRR condition at 14 dpi. n=4 independent conversions per condition. Mean+/−s.e.m. One-way ANOVA. (G) Effect of mCherry, Top2a-T2A-mCherry, or Top2a-T2A-mCherry+Top1 overexpression on percentage of Hb9::GFP+ iMNs at 14 dpi for 6FDD+RepSox condition. n=3-4 independent conversions per condition. Mean+/−s.e.m. Oneway ANOVA. (H) Effect of mCherry, Top2a-T2A-mCherry, or Top2a-T2A-mCherry+Top1 overexpression on percentage of Hb9::GFP+ and mCherry+double positive iMNs at 14 dpi for 6FDD+RepSox condition. n=3-4 independent conversions per condition. Mean+/−s.e.m. Oneway ANOVA. (I) Effect of Top1 overexpression on conversion yield of 6FDD with and without RepSox at 14 dpi. n=7-14 independent conversions per condition. Median+/−interquartile range. Kruskal-Wallis test. Significance summary: p>0.05 (ns), *p≤0.05, **p≤0.01, ***p≤0.001, and ****p≤0.0001.

FIG. 12A-Q shows Topoisomerase expression sustains transcription in S-phase, reduces R-loop formation and negative DNA supercoiling. (A) Representative images of MEFs treated with or without 100 μM bleomycin for 15 minutes. Scale bar represents 10 μM. (B) Mean intensity of biotinylated psoralen conjugated streptadvidin-Alexa Fluor 594 in MEFs treated with or without 100 μM bleomycin for 15 minutes. Mean intensity determined by total intensity in nuclear area as determined by Hoecsht and normalized to total nuclear area. n=60-64 cells from 3 independent conversions per condition. Median+/−interquartile range. Mann-Whitney test. (C) Mean intensity of biotinylated psoralen conjugated streptadvidin-Alexa Fluor594 at 4 dpi in 6FDDRR+Scrambled shRNA, shTop1-B, and shTop2a-B shRNA conditions at 4 dpi. n=99-162 cells from 3 independent conversions per condition. Median+/−interquartile range. Kruskal-Wallis test. (D) Relative amount of DNA protected by exonuclease digestion in region 500 bp upstream of transcription start site for Actb in Control-Puro cells treated with trimethylpsoralen with or without cross-linking at 4 dpi. Relative DNA protected by exonuclease digestion determined by psoralen intercalation in exonuclease-digested compared to nonexonuclease-digested control for each sample and measured with TMP-qPCR. n=3-4 independent transductions per condition. Mean+/−s.e.m. Unpaired t-test. (E) Reads from Actb, Gapdh, and Sod1 in 6F and 6FDDRR quantified by cell number normalized (CNN) RNAseq at 4 dpi. n=3 independent conversions per condition. Mean+/−s.e.m. Unpaired t-test between the two conditions for each gene. (F) Fraction of RNAPII ChIPseq peaks in TSS-proximal region (i.e. within 500 bp of transcription start site) in 6F and DDRR conditions at 4 dpi (left) and (right) genome browser track showing even distribution of RNAPII over Fxr1 gene body and reduced RNAPII in TSS-proximal region for DDRR compared to 6F. (G) Representative images of dot blot of S9.6 R-loop and ssDNA intensities for Control-Puro, 6F, and 6FDDRR at 4 dpi. n=6 independent transductions per condition. (H) Relative R-loop intensity quantified and normalized to ssDNA for Control-Puro, 6F, and 6FDDRR at 4 dpi. n=6 independent transductions per condition. Mean+/−s.e.m. One-way ANOVA. (I) Representative images of S9.6 immunofluorescence in 6F MEFs treated with buffer or RNAse H. (J) R-loop intensity per area excluding nucleoli in 6F MEFs treated with buffer or RNAse H. n=110-119 cells from 3 independent conversions per condition. Median+/−interquartile range. Mann-Whitney test. (K) R-loop intensity per area excluding nucleoli at 4 dpi in Control-Puro, 6F, and 6FDDRR conditions. R-loop intensity per area determined by staining with S9.6 antibody. n=118-163 cells from 3 independent conversions per condition. Median+/−interquartile range. Kruskal-Wallis test. (L) R-loop intensity per area at 4 dpi in 6FDDRR+shScrambled, shTop1-B, and shTop2a-B shRNAs. R-loop intensity per area determined by staining with S9.6 antibody. n=277-495 cells per condition. Median+/−interquartile range. Kruskal-Wallis test. (M) Representative images of stalled/terminated replication forks and new origins in DNA fiber labeling assay for 6F and 6FDDRR conditions at 4 dpi. Scale bar represents 10 μM. (N) Scatter plot showing EdU incorporation and DAPI via flow cytometry at 4 dpi in uninfected MEFs treated with DMSO or 10 μM Aphidicolin for 2 hr. (O) Percentage of cells in S-phase for Control-Puro, 6F, or 6FDDRR conditions measured at 4 dpi via EdU incorporation using flow cytometry. Percentage relative to all viable cells sorted based on FSC and SSC profile via FACS. S-phase determined by intensity above EdU incorporation in non-proliferative, irradiated MEFs (FIG. 10H). n=3-4 independent transductions per condition. Mean+/−s.e.m. One-way ANOVA. (P) Fraction of S-phase cells with high RNAPII activity measured via RNAPII Ser2p intensity at 4 dpi via flow cytometry. S-phase determined as previously described. High RNAPIISer2p defined as the top quartile of RNAPIISer2p intensity in Control-Puro infected cells in S-phase cells. n=4 independent transductions per condition. Mean+/−s.e.m. One-way ANOVA. (Q) Percentage of cells in S-phase with high RNAPII activity at 4 dpi for 6FDDRR+Scrambled, 6FDDRR+Top1-B, and 6FDDRR+Top2a-B shRNAs measured at 4 dpi via EdU incorporation and RNAPII Ser2p intensity measured by flow cytometry. Percentage of cells in S-phase with high RNAPII activity calculated based on total number of cells. n=4 independent transductions per condition. Mean+/−s.e.m. One-way ANOVA. Significance summary: p>0.05 (ns), *p≤0.05, **p≤0.01, ***p≤0.001, ****p≤0.0001.

FIG. 13A-C shows Converting HHCs adopt the induced motor neuron transcriptional program, accelerating maturation. (A) tSNE plot with clusters of iMNs identified by neuronal gene signature and other Hb9::GFP+ cells captured at 14 dpi in reprogramming “6F”, “6FDDRR” and “Top1+6FDDRR” clusters comprise cells with non-neuronal gene expression profiles. (B) Gene expression for clusters in (A) separate neuronal iMN clusters with high Map2 (middle) expression from non-neuronal clusters with high Col1a1 (top) and lower Isl1 (bottom) expression. (C) Representative images of Hb9::GFP+ multipolar 6F (top) or 6FDDiMNs (bottom) at 14 dpi. Scale bar represents 5 M for 6F-iMN and 10 M for 6FDD-iMN.

FIG. 14A-D demonstrates that the DDRR cocktail boosts reprogramming across multiple cell types and species. Related to FIG. 7. (A) Percent HHCs in tail-tip fibroblasts in Control-Puro, 6F, and 6FDDRR at 4 dpi measured by flow cytometry. Hyperproliferating cells were defined as cells showing a two-fold increase in division rate (i.e. an eight-fold decrease in CFSE intensity) compared to the average of the control population, which was comprised of Control-Puro MEFs. n=4 independent transductions per condition. Mean+/−s.e.m. One-way ANOVA. (B) Representative images of multipolar human 7F (left) or 7FDD-iMNs (right) at 35 dpi. Scale bar represents 10 M. (C) Average current versus voltage curve of outward potassium channels in 7F or 7FDD human iMNs at 35 dpi. (D) Average current versus voltage curve of sodium channel in 7F or 7FDD human iMNs at 35 dpi. Significance summary: p>0.05 (ns), *p≤0.05, ** p≤0.01, ***p≤0.001, ****p≤0.0001.

### DETAILED DESCRIPTION

As used herein and in the appended claims, the singular forms “a,” “an,” and “the” include plural referents unless the context clearly dictates otherwise. Thus, for example, reference to “a derivative” includes a plurality of such derivatives and reference to “a subject” includes reference to one or more subjects and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

The publications discussed throughout the text are provided solely for their disclosure prior to the filing date of the present application. Nothing herein is to be construed as an admission that the inventors are not entitled to antedate such disclosure by virtue of prior disclosure.

Cellular reprogramming redirects the transcriptional state of a cell to a new fate. By supplying inaccessible somatic cell types in unique genomic contexts, transcription factor-mediated reprogramming massively expands the potential for in vitro disease modeling. However, epigenetic barriers limit reprogramming between somatic lineages to rare events and cause incomplete conversion of gene regulatory networks (GRNs). Efforts to identify epigenetic factors limiting reprogramming have focused primarily on induced pluripotent stem cell (iPSC) generation, and many of these findings are specific to iPSC reprogramming.

Provided herein is the identification of universal roadblocks to reprogramming that extend beyond iPSCs into other lineages and strategies that can be used to overcome them. To this end, systems-level constraints limiting the conversion of fibroblasts into motor neurons, as well as other paradigms, were examined. In the studies presented herein, it was found that the addition of the reprogramming factors sharply increased the transcription rate in cells and reduced the rate of DNA synthesis and cell division, highlighting the existence of trade-offs between transcription and cell replication during the conversion process. Most cells display either a high rate of transcription and limited proliferation or a high rate of proliferation and limited transcription, with both cell states being refractory to reprogramming. A privileged population of cells were identified herein that were capable of both high proliferation and high transcription rates which contributed to the majority of reprogramming events. This indicates that a high rate of proliferation is not sufficient for efficient reprogramming and that it must be coupled with high rates of transcription. Using a cocktail of genetic and chemical factors (DDRR cocktail) allowed for the expansion of the hypertranscribing, hyperproliferating cell (HHC) population and achieved induced motor neuron reprogramming at near-deterministic rates. This approach was found to be effective across all starting, targeted cell types tested. Transcription and DNA synthesis interfere directly through collisions of transcription and replication machinery, as well as indirectly by generating inhibitory DNA structures and topologies (e.g., A-loops and supercoiling). In the studies presented herein, topoisomerases were found as key regulators for the emergence and expansion of privileged HHCs. By expanding the population of HHCs, the maturation of the resulting cells was accelerated and the heterogeneity was also reduced. Thus, use of the DDRR cocktail of the disclosure overcame molecular barriers to reprogramming by suppressing biophysical constraints that govern transcription and replication processes.

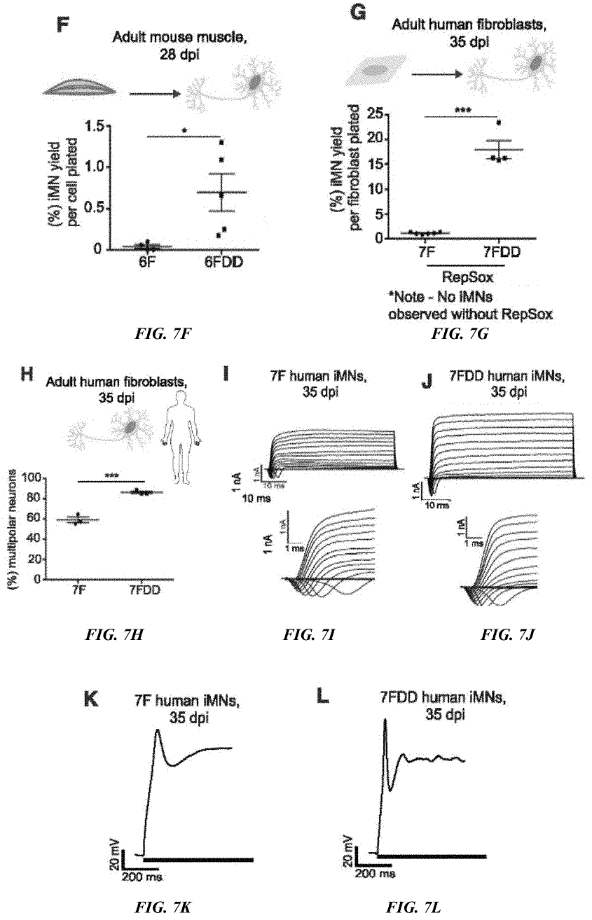

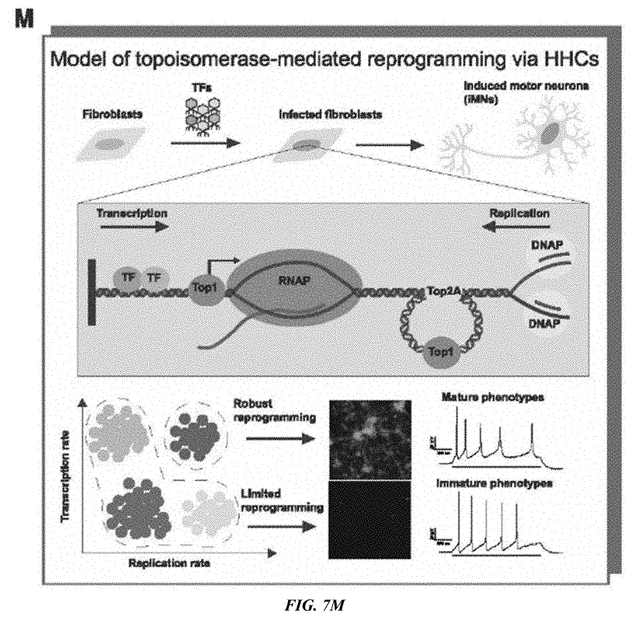

The studies presented herein, identified that hypertranscription and hyperproliferation were a central driver of reprogramming, and which could overcome molecular barriers to lineage conversion across multiple species and somatic cell states. Combined hypertranscription and hyperproliferation is rare because transcription and proliferation antagonize each other during reprogramming. Forced expression of the reprogramming transcription factors increases genomic stress in the form of A-loops, DNA torsion, and reduced processivity of DNA replication forks. Consequently, reprogramming remains restricted to rare cells with high transcriptional and proliferative capacity that reprogram at near-deterministic rates. By introducing chemical and genetic perturbations that mitigate antagonism by activating topoisomerases, the capacity for high rates of coincident transcription and proliferation extend conversion to otherwise un-reprogrammable cells (see FIG. 7M). Cells exhibiting combined hypertranscription and hyperproliferation are also capable of achieving greater functional maturity in the reprogrammed state, indicating that increasing the cell's capacity to balance trade-offs during conversion can surmount maturity barriers. The studies presented herein suggests that the enhanced design of reprogramming vectors to account for limitations in cellular hardware may improve the predictability and determinism of reprogramming.

As used herein “dedifferentiation” signifies the regression of lineage committed cell to the status of a stem cell, for example, by “inducing” a de-differentiated phenotype. For example, as described further herein KLF4, OCT4, SOX2, c-MYC or n-MYC or L-MYC, GLIS1 and/or Nanog can induce de-differentiation and induction of mitosis in lineage committed mitotically inhibited cells.

“Differentiation” refers to he progression of lienage committed cells to the status of a fully differentiated or somatic cell type.

“Reprogramming” includes dedifferentiation and differentiation of a cell type to a less committed lineage or more committed lineage respectively.

As described herein, the compositions and methods of the disclosure provide the ability obtain cells that are capable of reprogramming. Such compositions and methods are useful for obtaining cells to de-differentiate to form stem cells (e.g., induce the formation of stem cells). Stem cells are cells capable of differentiation into other cell types, including those having a particular, specialized function (e.g., tissue specific cells, parenchymal cells and progenitors thereof). There are various classes of stem cells, which can be characterized in their ability to differentiate into a desired cell/tissue type. For example, “progenitor cells” can be either multipotent or pluripotent. Progenitor cells are cells that can give rise to different terminally differentiated cell types, and cells that are capable of giving rise to various progenitor cells. The term “pluripotent” or “pluripotency” refers to cells with the ability to give rise to progeny cells that can undergo differentiation, under the appropriate conditions, into cell types that collectively demonstrate characteristics associated with cell lineages from all of the three germinal layers (endoderm, mesoderm, and ectoderm). Pluripotent stem cells can contribute to all embryonic derived tissues of a prenatal, postnatal or adult animal. A standard art-accepted test, such as the ability to form a teratoma in 8-12 week old SCID mice, can be used to establish the pluripotency of a cell population; however identification of various pluripotent stem cell characteristics can also be used to detect pluripotent cells. “Pluripotent stem cell characteristics” refer to characteristics of a cell that distinguish pluripotent stem cells from other cells. The ability to give rise to progeny that can undergo differentiation, under the appropriate conditions, into cell types that collectively demonstrate characteristics associated with cell lineages from all of the three germinal layers (endoderm, mesoderm, and ectoderm) is a pluripotent stem cell characteristic. Expression or non-expression of certain combinations of molecular markers are also pluripotent stem cell characteristics. For example, human pluripotent stem cells express at least some, and in some embodiments, all of the markers from the following non-limiting list: SSEA-3, SSEA-4, TRA-1-60, TRA-1-81, TRA-2-49/6E, ALP, Sox2, E-cadherin, UTF-1, Oct4, Rex1, and Nanog. Cell morphologies associated with pluripotent stem cells are also pluripotent stem cell characteristics. In comparison, a multipotent stem cell is capable of differentiating into a subset of cells compared to a pluripotent stem cell. For example, a multipotent stem cell may be able to undergo differentiation into one or two of the three germinal layers. As used herein, “non-pluripotent cells” refer to mammalian cells that are not pluripotent cells. Examples of such cells include differentiated cells as well as multipotent cells. Examples of differentiated cells include, but are not limited to, cells from a tissue selected from bone marrow, skin, skeletal muscle, fat tissue and peripheral blood. Exemplary cell types include, but are not limited to, fibroblasts, hepatocytes, myoblasts, neurons, osteoblasts, osteoclasts, and T-cells.

In a particular embodiment, the disclosure provides for a DDRR cocktail that can be used in methods described herein for forming a population of hypertranscribing, hyperproliferating cells (HHCs). The DDRR cocktail comprises at least a TGF-β inhibitor, and a dominant negative p53 mutant. The DDRR cocktail may advantageously further comprise a Ras mutant. Use of the DDRR cocktail disclosed herein increased cellular reprogramming efficiency about 100-fold to near-deterministic rates in mouse and human cells.

The term “TGFβ signaling pathway” as used herein refers to downstream signaling events attributed to TGFβ and TGFβ like ligands. Engagement of Type II TGFβ receptors, for example, by a TGFβ ligand leads to the recruitment of Type I TGFβ receptors, which form heterodimers with Type II TGFβ receptors. Upon heterodimer formation, the Type I receptor is phosphorylated, which in turn phosphorylates and activates the SMAD family of proteins, thereby triggering a TGFβ signaling cascade. The signaling cascade ultimately leads to altered regulation of the expression of mediators involved in a variety of cellular processes, including, without limitation, cell growth, cell differentiation, tumorigenesis, apoptosis, and cellular homeostasis.

The term “inhibitor of the TGFβ signaling pathway” as used herein refers to inhibition of at least one of the proteins involved in the signal transduction pathway of TGFβ. Such inhibitors of the TGFβ signaling pathway encompass, for example, a TGFβ receptor inhibitor (e.g., a small molecule, an antibody, an siRNA), a TGFβ sequestrant (e.g., an antibody, a binding protein), an inhibitor of receptor phosphorylation, an inhibitor of a SMAD protein, or a combination of such agents.

In one embodiment, the TGFβ signaling pathway inhibitor comprises or consists essentially of a TGFβ receptor inhibitor. Assays for testing a compound to determine if it inhibits TGFβ receptor signaling are known in the art and are a matter of routine practice. Such assays may, for example, include determinations of phosphorylation status of the receptor or expression of downstream proteins controlled by TGFβ in cells cultured in the presence of the compound and comparing these determinations to those made for cells not treated with a TGFβ receptor inhibitor. An agent is identified as a TGFβ signaling pathway inhibitor if the level of phosphorylation of the Type I TGFβ receptor in cells cultured in the presence of the agent is reduced by at least 20%, at least 30%, at least 40%, at least 50%, at least 60%, at least 70%, at least 80%, at least 90%, at least 95%, at least 99%, or even 100% (no phosphorylation) relative to the level of phosphorylation of the Type I TGFβ receptor in cells that are cultured in the absence of a TGFβ signaling pathway inhibitor.

As used herein, the term “TGFβ/Activin/Nodal signaling inhibitor” refers to a small molecule or protein modulator that is capable of downregulating signaling along the transforming growth factor beta (TGFβ/Activin/Nodal signaling pathway. In certain embodiments, the TGFβ/Activin/Nodal signaling inhibitor directly targets TGFβ type 1 receptor (TGFβ R1), also known as activin receptor-like kinase 5 (ALK5). Inhibitors of TGFβ receptor activity encompassed herein include, without limitation, an antibody, a small molecule, or an RNA interference molecule capable of inhibiting a TGFβ signaling pathway or combinations thereof. Exemplary inhibitors of TGFβ receptor activity also include the following compounds: A 83-01, D 4476, GW 788388, LY 364947, R 268712, RepSox, SB 431542, SB 505124, SB 525334, and SD 208. Such agents are commercially available and can, for example, be purchased from Sigma, Tocris, Fisher, and Biovision.

Examples of TGF-β inhibitors that can be used in the cocktail include, but are not limited to, RepSox, SB431542, A-83-01, LY-364947, LY2157299, LY-364947, A 83-01, and ALK5 inhibitor. In a particular embodiment, the DDRR cocktail comprises the TGF-β inhibitor, RepSox.

A dominant negative p53 mutant generally lacks a DNA-binding domain. An example of such dominant negative p53 mutant, includes p53DD.

An example of a Ras mutant includes hRAS G12V.

In the studies presented herein, the DDRR cocktail of the disclosure relieved DNA supercoiling by activating topoisomerases. The DDRR cocktail of the disclosure, or parts thereof, such as TGF-beta inhibitors, or topoisomerase overexpression or activation, can be used for one or more of the following:

to increase cell reprogramming in vivo for regenerating lost tissues;

to increase the efficiency of cell reprogramming in vitro and the maturity of the reprogrammed cells to enable new types of human disease models; and/or

to increase the efficiency of cell reprogramming of adult cells or cells with special epigenetic marks, avoiding the erasure of epigenetic marks that normally occurs with iPSC reprogramming. The retention of these epigenetic marks could be useful for in vitro studies, including studies of aging or age-dependent diseases.

Suitable sources of cells can include any somatic cell. In one embodiment, a useful cell type for is a fibroblast that can be contacted with a cocktail and using the methods of the disclosure to obtain HHC fibroblast cells. Fibroblasts may be readily isolated by disaggregating an appropriate organ or tissue which is to serve as the source of the fibroblasts. This may be readily accomplished using techniques known to those skilled in the art. For example, the tissue or organ can be disaggregated mechanically and/or treated with digestive enzymes and/or chelating agents that weaken the connections between neighboring cells making it possible to disperse the tissue into a suspension of individual cells without appreciable cell breakage. Enzymatic dissociation can be accomplished by mincing the tissue and treating the minced tissue with any of a number of digestive enzymes either alone or in combination. These include but are not limited to trypsin, chymotrypsin, collagenase, elastase, and/or hyaluronidase, DNase, pronase, dispase etc. Mechanical disruption can also be accomplished by a number of methods including, but not limited to, the use of grinders, blenders, sieves, homogenizers, pressure cells, or insonators to name but a few. For a review of tissue disaggregation techniques, see Freshney, Culture of Animal Cells. A Manual of Basic Technique, 2d Ed., A.R. Liss, Inc., New York, 1987, Ch. 9, pp. 107-126.

Once the tissue has been reduced to a suspension of individual cells, the suspension can be fractionated into subpopulations from which the fibroblasts and/or other stromal cells and/or elements can be obtained. This also may be accomplished using standard techniques for cell separation including, but not limited to, cloning and selection of specific cell types, selective destruction of unwanted cells (negative selection), separation based upon differential cell agglutinability in the mixed population, freeze-thaw procedures, differential adherence properties of the cells in the mixed population, filtration, conventional and zonal centrifugation, centrifugal elutriation (counterstreaming centrifugation), unit gravity separation, countercurrent distribution, electrophoresis and fluorescence-activated cell sorting. For a review of clonal selection and cell separation techniques, see Freshney, Culture of Animal Cells. A Manual of Basic Techniques, 2d Ed., A.R. Liss, Inc., New York, 1987, Ch. 11 and 12, pp. 137-168.

The isolation of fibroblasts may, for example, be carried out as follows: fresh tissue samples are thoroughly washed and minced in Hanks balanced salt solution (HBSS) in order to remove serum. The minced tissue is incubated from 1-12 hours in a freshly prepared solution of a dissociating enzyme such as trypsin. After such incubation, the dissociated cells are suspended, pelleted by centrifugation and plated onto culture dishes. All fibroblasts will attach before other cells, therefore, appropriate stromal cells can be selectively isolated and grown.

In some embodiments, HHC cells are isolated by culturing the cells with a cocktail of the disclosure followed by isolating cells having a hypertranscription and hyperproliferative phenotype. These HHC cells can then be banked (tissue banked) or used for reprogramming. In the case of dedifferentiation, the cells treated under conditions such that the dedifferentiate (e.g., are transfected with a vector expression one or more reprogramming factors selected from Oct4, Sox2, Klf4, cMyc, Glis1, Nanog and Lin28). The reprogramming vectors can be delivered using various methods in the art (e.g., alpha viruses, lentiviruses, AAV viruses, naked DNA etc.).

It is to be understood that while the disclosure has been described in conjunction with specific embodiments thereof, that the foregoing description as well as the examples which follow are intended to illustrate and not limit the scope of the disclosure. Other aspects, advantages and modifications within the scope of the disclosure will be apparent to those skilled in the art to which the disclosure.

### EXAMPLES

Cell Lines and Tissue Culture. HEK293, Plat-E, mouse embryonic fibroblasts, and primary human fibroblasts were cultured in DMEM supplemented with 10% FBS at 37° C. with 5% CO₂. Mouse tail tip fibroblasts were cultured in DMEM supplemented with 40% FBS at 37° C. with 5% CO₂. The following are the sex of primary human fibroblasts used in this study: Foreskin fibroblasts (BJ)—male, adult fibroblasts (GM05116)—female.

Isolating fibroblasts and cell culture. Hb9::GFP-transgenic mice (Jackson Laboratories) were mated with C57BU6 mice (Jackson Laboratories) and MEFs were harvested from Hb9::GFP E12.5-E13.5 embryos under a dissection microscope (Nikon SMZ 1500). To eliminate contaminating neurons, the head and spinal cord were removed. The fibroblasts were passaged at least once before being used for experiments. Neonatal mesenchymal cells were harvested by collagenase I digestion of hind limb muscle. Human adult fibroblasts were obtained from Coriell (GM05116). Irradiated MEFs were obtained from GlobalStem (Cat. No: GSC-6001G).

Plasmid construction. Retroviral and lentiviral plasmids were constructed by Gateway and Gibson cloning into entry vectors pDONR221 or pENTR4-DS. Entry clones were recombined into destination vectors via LR reaction into the pMXS-DEST (retro) FUWO-tetO-DEST (lent).

Viral transduction and iMN reprogramming. Retroviral transduction and iMN reprogramming from MEFs was performed. Briefly, retroviral transduction was performed using Plat-E retroviral packaging cells (Cell Biolabs, Inc., RV-101). MEFs were transduced twice with Ascl1, Bm2, lsl1, Lhx3, Mytl1, and Ngn2 or a polycistronic NIL construct at 48 and 72 hours after transfection. For human iMN experiments, retroviruses were generated in 293T cells and co-transfected with pLK and pHDMG packaging plasmids, and NEUROD1 was added to the reprogramming cocktail. In experiments in which iMN formation was quantified by microscopy or iMNs were functionally evaluated using electrophysiology, mixed glia isolated from P2 ICR mouse pups were added to infected fibroblasts 2 days after transduction. It is important to note that in other experiments, including those in which the number of hyper-proliferating or hyper-transcribing cells were analyzed, CFSE labeling was used, FACS sorting was used prior to iMN formation, or FACS sorting was used to quantify the number of iMNs out of total viable cells at the end of reprogramming, glia were omitted from the glia media added to the reprogramming cultures in order to avoid confounding the results. The day after glia media addition, medium was switched to complete N3 neuronal medium (DMEM/F-12 (VWR) with N2 and B27 (Thermo Fisher) supplements and 1% glutamax (Thermo Fisher)). Medium was supplemented with neurotrophics, GDNF, BDNF, and CNTF (R&D Systems) and FGF-Basic (Peprotech), each at 10 ng/mL. When included for reprogramming, RepSox (Selleck) was added to N3 media at a final concentration of 7.5 uM.

iHC reprogramming. Retroviral transduction of MEFs was performed using Plat-E retroviral packaging cells (Cell Biolabs, Inc., RV-101). Atoh1::nGFP MEFs were transduced with Atoh1, Bm3c, and Gfi1 at 48 and 72 hours post-transfection. Two days after transduction, media was changed to induced hair cell media (DMEM/F-12+N2+B27) supplemented with FGF-Basic (Peprotech) and H B-EGF (Peprotech) and final concentrations of 2.5 ng/uL and 5 ng/uL, respectively.

IDAN and IN reprogramming. Retroviral transduction of non-transgenic or Tau::GFP MEFs was performed using Plat-E. MEFs were transduced with Ascl1, Bm2, Myt1 FoxA2, and Lmx1a for iDAN reprogramming. Alternately, MEFs were transduced with Asc/1, Bm2, and Mytl1 for iN reprogramming. Two days after transduction, mixed glia isolated from P2 ICR mouse pups were added to converting cultures. The next day, complete neuronal N3 media with neurotrophic factors (FGF, GDNF, CNTF, and BDNF at each at 10 ng/mL was added to converting cultures.

shRNA mediated knockdown of Mbd3, Gatad2a, Top1, Top2a, Mbd3, Gatad2a, Chd4, Top1 and Top2a shRNA lentiviral constructs were obtained from Sigma. Lentiviruses were generated in 293T cells and packaged via co-transfection with pPax2 as well as VSVG envelope using PEI transfection reagent. Hb9::GFP+ MEFs were co-transduced with scrambled, Mbd3, Gatad2a, Top 1, or Top2a shRNAs during the second day of PlatE transduction with the motor neuron factors.

qRT-PCR quantification of shRNA-mediated Mbd3, Gatad2a, Top1, Top2a knockdown. For experiments measuring knockdown of Mbd3, Gatad2a, Top 1 or Top2a, cells were collected 4 days after transduction with motor neuron factors and shRNAs. RNA isolation was performed using TRIzol LS reagent according to the manufacturer's instructions (Thermo Fisher Cat. No: 10296010). Reverse transcription of purified RNA was performed using random hexamer primers and New England Biolabs protoscript first strand cDNA synthesis (VWR Cat. No: 101640-908). qPCR was performed using primers tor Mbd3, Gatad2a, Top1 and Top2a and iTaq universal SYBR green (Bio-Rad Cat. No: 1725125). The following primer sequences for endogenous Mbd3, Gatad2a, Top1 and Top2a genes were used:

Mbd3
(5′-TCCAGGTCTCAGTGCAGGGA (SEQ ID NO: 1) and 5′-
TGACTTCCTGGTGGGCTGC (SEQ ID NO: 2),
Gatad2a
(5′-AATAACGGGTCCTCACTACAG (SEQ ID NO: 3) and 5′-
GTATTCTCGCTGTCGATCCA (SEQ ID NO: 4)),
Top1
(5′-TCTCTAGTCCGCCACGAATTA (SEQ ID NO: 5) and 5′-
CATCTCGAAGCCTCTTCAATGG (SEQ ID NO: 6))
and
Top2a
(5′-GCTCCTCGAGCCAAATCTGA (SEQ ID NO: 7) and 5′-
CTACCTATAAAACTGGCTCCGT (SEQ ID NO: 8)).

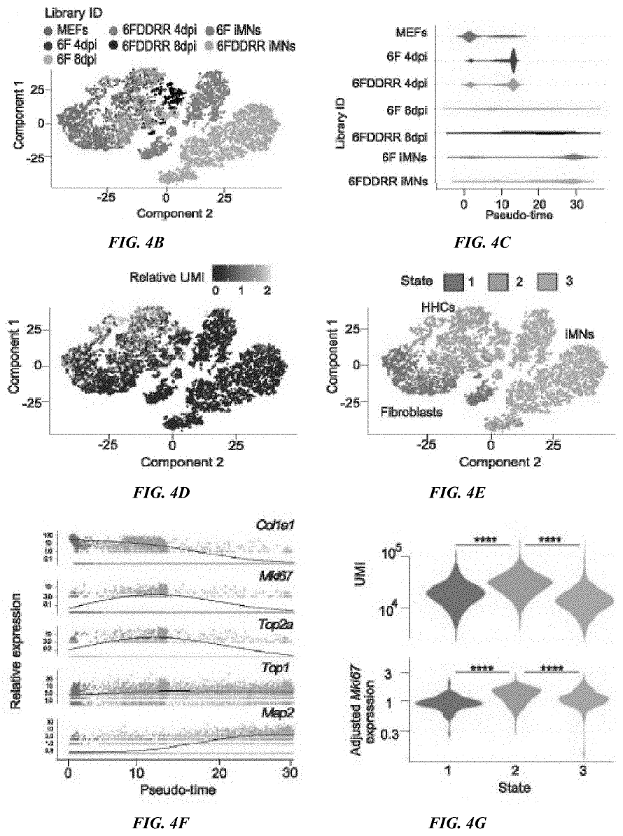

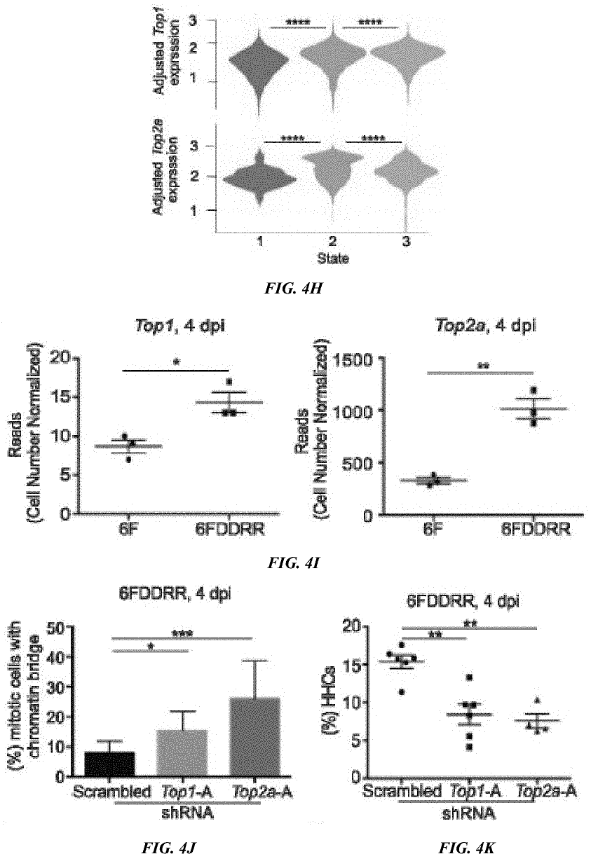

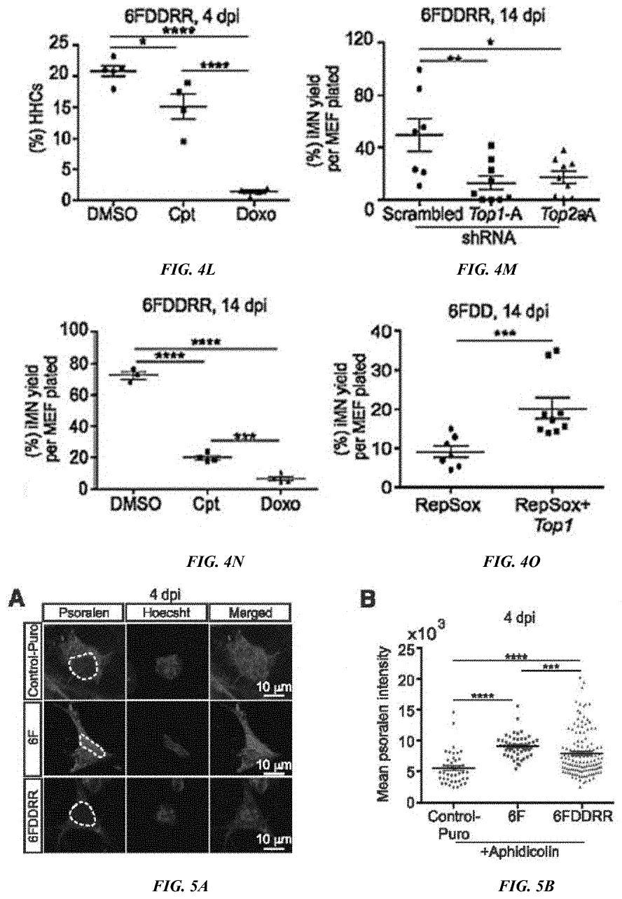

Quantification of Conversion Yield. All reprogrammed cultures were imaged using either the Biostation CT or Molecular Devices Image Express and manually quantified using Fiji. Yield of converted cells was calculated as the number of cells with the proper morphology and marker(s) on the final day of conversion over the number of cells seeded for conversion. For iMNs, the number of Hb9::GFP+ MEF− or explant-derived cells with neuronal morphologies was quantified between 14-17 dpi. In the single cell RNA sequencing experiments in FIG. 4, iMNs were collected at 14 dpi. For iDANS and iNs, Tau::GFP+ or Map2+ cells with neuronal morphologies were manually quantified between 17 dpi. For iHCs, the number of Atoh1::nGFP+ cells was used to quantify percent iHCs between 17 dpi. For iMNs, adult human fibroblast-derived Map2+/DsRed or p53DD-T2A-RFP+ double-positive cells with neuronal morphologies were manually quantified between 35 dpi.

Whole cell patch clamp electrophysiology. Whole cell membrane potential and current recordings in voltage- and current-clamp configurations were made using an EPC9 patch clamp amplifier controlled with PatchMaster software (HEKA Electronics). Voltage- and current-clamp data was acquired at 50 kHz and 20 kHz, respectively, with a 2.9 kHz low-pass Bessel filter. For experiments, culture media was exchanged with warm extracellular solution consisting of in mM): 140 NaCl, 2.8 KCl, 10 HEPES, 1 MgCl₂, 2 CsCl₂, and 10 glucose, with pH adjusted to 7.3 and osmolarity adjusted to 310 mOsm. Glass patch pipettes were pulled on a Narishige PC-10 puller and polished to 5-7 MΩ resistance. Pipettes were also coated with Sylgard 184 (Dow Corning) to reduce pipette capacitance. The pipette solution consisted of in (mM): 130 K-gluconate, 2 KCl, 1CaCl₂, 4 MgATP, 0.3 GTP, 8 phosphocreatine, 10 HEPES, 11 EGTA, adjusted to pH 7.25 and 300 mOsm. Pipettes were sealed to cells in GΩ-resistance whole cell configuration, with access resistances typically between 10-20 MΩ, and leakage currents less than 100 pA. Capacitance transients were compensated automatically through software control. For current-voltage (IV) curves, cells were held in voltage clamp configuration at −70 mV and stepped through depolarizing voltages from −70 to 100 mV. A P/4 algorithm was used to subtract leakage currents from the traces. For action potential measurements, cells were held in current clamp configuration at 0 pA and action potentials were evoked by injecting positive depolarizing currents for 1 s. SFA ratios were calculated as the time interval between the first two APs evoked to the time interval between the last two APs evoked using the lowest current injection that generated APs. Measurements were taken at room temperature (approximately 20-25° C.). Data was analyzed and plotted in Igor Pro (WaveMetrics).

CFSE cell labeling to measure cellular proliferation. One day after retroviral infection, fibroblasts were labeled with CellTrace CFSE Cell Proliferation Kit GFP (Invitrogen, Cat. No: C34554) or Far Red (Invitrogen, Cat. No: C34572) at a final concentration of 10 uM. Briefly, media was removed, CFSE added to the cells, and incubated at 37° C. for 30 minutes. After incubation cells were washed once with PBS, then replaced with fresh media. Generally, cells were harvested for FACS sorting 72 hr following labeling without addition of glial cultures. Fast cycling cells were determined by examining the distribution from cells infected with reprogramming factors. During reprogramming, the dimmest 15% of cells in 6F conditions at 4 dpi were used to set the gate for fast-cycling cells. Cells with lower CFSE intensity were gated as fast-cycling. For all re-plating experiments, gates were set using the dimmest 15% of cells in 6F conditions. Generally, it was found that the absolute CFSE intensity of the fast-cycling cells was 8-fold lower than mean CFSE of the entire population, indicating three more divisions over 72 hr. With a putative average 24 h cell cycle, cells divide 3 times over 72 hours, while fast cells divide 6 times or more, suggesting a <12 h cell cycle.

Chromatin immunoprecipitation (ChiP)-sequencing for RNA Poll. One day after addition of N3 media (and without addition of glial cultures), cells were fixed by adding fresh formaldehyde to culture media at a 1:10 volume (11% final concentration) and fixed for 15 minutes at room temperature with agitation. Formaldehyde was quenched with by adding a glycine solution to cells at 1:20 volume (2.5 M final concentration) and incubated at room temperature for 5 minutes. Cells were then scraped from cell dish, collected into 1.5 mL Eppendorf tubes, and kept on ice for the remainder of processing. Cells were spun down at 800×g for 10 minutes at 4° C. After pelleting, supernatant was removed and cells were resuspended in 1 mL chilled 0.5% lgepal in PBS, triturating each cell sample by pipetting up and down several times. Samples were then spun down for 10 minutes at 800×g at 4° C. After spinning down, cells were again resuspended in 1 ml 0.5% lgepal in PBS and 1 uL PMSF was added (final concentration of 100 mM). Cell pellets were then snap-frozen and stored at −80° C. Cells were processed by Active Motif via a standard ChiPseq protocol to enrich for RNAPII bound regions of DNA. Replicate RNA Pol II ChiP reactions were performed using 25 pg of primary MEF, 6F, and DDRR chromatin and 4 μg of Abflex RNAPII antibody (Active Motif, cat #91151). Libraries were generated via standard Ilumina protocols and sequenced to generate 30M reads per sample. The 75-nt sequence reads generated by Ilumina sequencing (using NextSeq 500) are mapped to the mm 10 genome using the BWA algorithm with default settings. Sicer was used to call peaks of enrichment resulting in 20,000 peaks per sample. Peaks called within 500 bp of a transcription start site were deemed “TSS-proximal peaks.”

Cleaved caspase-3, mKi67, RNA PolIII immunolabeling for FACS sorting. One day after addition of N3 media (and without addition of glial cultures), cleaved caspase-3 and mKi67 labeling and subsequent FACS sorting for analysis was performed. Cells were trypsinized with 0.25% Trypsin-EDTA (Genesee Scientific), resuspended, and then spun down. Cells were then fixed in 4% paraformaldehyde for 15 minutes at room temperature in the dark. Cells were washed with PBS, pelleted, and permeabilized with 0.5% Triton X-100 for 15 minutes at room temperature in the dark. After permeabilization, cells were blocked in 3% FBS in PBS block solution for 30 minutes at room temperature in the dark with rotation. After being spun down, cells were then incubated in primary antibodies (1:200 dilution in 3% block solution) for 45 minutes at room temperature with rotation. Cells were washed with block solution, spun down, and then incubated in secondary antibodies (1:200 dilution in 3% block solution) for 30 minutes at room temperature in the dark with rotation. Cells were then washed in block solution, spun down, and resuspended in 150-200 11l PBS containing DAPI (100×) prior to FACS sorting and analysis. The following primary antibodies were used: rabbit anti-cleaved caspase-3 (Abcam Cat No: ab13847), rabbit anti-Ki67 (GeneTex GTX16667 Cat. No: 89351-224) and rabbit anti-RNA polymerase II CTD repeat YSPTSPS (phospho 52) (Abcam Cat No: ab5095).

DNA Fiber Assay. One day after addition of N3 media (and without glial cultures), cells were pulse-labeled with ldU (50 μM) and CldU (100 μM final concentration) for 20 and 30 minutes, respectively at 37° C. Cells were washed with PBS, trypsinized with 0.25% Trypsin-EDTA and spun down. Cells were resuspended in 50 μL, put on ice, and resuspended to a concentration of 400 cells/4 in PBS. Three, 2 μL aliquots of each cell sample was spotted onto silane-coated slides and tilted to allow the cells to streak across the slide lengthwise. The cell preparations were dried for ˜15-20 minutes and then lysed (1M Tris pH 7.4+0.5M EDTA+10% SDS in ddH₂O). DNA spreads were air-dried for 12 hours at room temperature and then fixed in methanol:acetic acid (3:1) for 2 minutes at room temperature. Slides were dried overnight at room temperature protected from light and then stored at −20° C. for at least 24 hours before antibody labeling. The fiber spreads were treated with 2.5M HCl for 30 minutes and then blocked in 5% BSA for 30 minutes in a “humidified chamber.” Fiber spreads were incubated with mouse α-BrdU (1:500, to detect IdU) and rat α-BrdU (1:500, to detect CIdU) primary antibodies for 1 hour at room temperature and then incubated for 15 minutes in stringency buffer (1M Tris pH 7.4+5M NaCl+10% Tween+10% NP40 in ddH₂O). Slides were blocked again for 30 minutes and then incubated with rabbit α-mouse 594 (1:1000) and chicken α-rat 488 (1:750) secondary antibodies for 30 minutes at room temperature. After washes in 0.1% Tween in PBS, slides were blocked again at room temperature and then incubated with goat α-rabbit 594 (1:1000) and goat α-chicken 488 (1:750) tertiary antibodies for 30 minutes at room temperature. After a wash with 0.1% Tween in PBS followed by PBS washes, glass coverslips were mounted onto the silane slides using Antifade. The following primary antibodies were used: Monoclonal anti-IdU antibody produced in mouse (Sigma Cat. No: SAB3701448-100UG) and anti-BrdU antibody (BU1/75 (ICR1)) detects CIdU (Abcam Cat. No: ab6326).

DNA-RNA Hybrid R-loop Staining and RNase Treatment. One day after addition of N3 media (and without addition of glial cultures), cells were fixed in 4% paraformaldehyde for one hour at 4° C. in the dark. Cells were then permeabilized in 0.2% Triton X-100 in PBS for one hour in the dark. Coverslips were then split into two and 1 half was used for RNase H treatment. Briefly, coverslip halves were treated with 250 μL of 1× buffer+2 μL RNase H at 37° C. for 36 hours prior to proceeding with antibody labeling. Then, all coverslips were incubated in 2% BSA in PBS block solution for 1 hour at room temperature. Cells were then incubated in primary antibodies (1:1000 nucleolin to label nucleoli+ 1:200 S9.6 to label DNA-RNA R-loops in 2% block solution) for 1 hour at room temperature followed by two PBS washes. Then, cells were stained with secondary antibodies (1:500 dilution in 2% block solution) for 2 hours at room temperature in the dark. After two PBS washes, cells were stained with Hoescht (1:1000) for 10 minutes at room temperature in the dark, washed again, and mounted onto glass slides using lmmuMount (ThermoFisher). The following primary antibodies were used: DNA-RNA R-loop S9.6 antibody (Kerafast Cat. No: ENH001), and nucleolin (Abcam Cat. No: ab22758).

Dot Blot far R-loop Analysis. For each sample, genomic DNA was purified from one well of a 6-well dish using the DNeasy Kit from QIAGEN. Samples were eluted using 150 μLs of elution buffer. Samples were then ethanol precipitated and resuspended in 7-10 μLs of water. 1 μL of each sample was spotted onto a positively charged nylon membrane (GE Healthcare) and dried for 10 minutes before cross-linking by exposure to 254 nm light for 3 minutes. Membranes were then blocked with 5% milk/TBST (20 mM Tris-HCl, 150 mM NaCl, 0.05% Tween 20, pH 7.5) for 1 h at room temperature. If RNase H treatment was performed, the membrane was incubated in 11 mL of 1× RNase H buffer with 44 μL of RNase H (New England Biolabs, Cat. No: M0297L) at 37° C. for 36 hours. Membranes were then washed twice with 5% milk/TBST S9.6 (1:1000, Kerafast Cat. No: ENH001) or single-stranded DNA (1:10,000, Millipore Cat. No: MAB3868) antibodies were added in 1% BSA/TBST and incubated at 4° C. overnight. For DNA that was going to be probed with the single-stranded DNA antibody, samples were heat denatured at 95° C. for 10 minutes and snap-cooled on ice for 2 minutes prior to spotting on the membrane. Membranes were then washed twice with TBST and probed with an anti-mouse horseradish peroxidase-linked anti body (1:5,000, Cell Signaling Cat. No: 7076S) for one hour at room temperature. Membranes were exposed using either the Amersham ECl Western Blotting Detection Kit (GE Healthcare, Cat. No: RPN21 08) or the SuperSignal West Femto Maximum Sensitivity Substrate (ThermoFisher Scientific Cat. No: 34577).

EU Incorporation for FACS Sorting. One day after addition of N3 media (and without addition of glial cultures), EU incorporation assays were performed according to manufacturer's instructions modified for FACS sorting (Invitrogen, Cat. No: C10330). Cells were incubated with 1 mM EU for 1 hour at 37° C., washed once with PBS, dissociated with 0.25% Trypsin-EDTA (Genesee Scientific), resuspended, and then spun down. Cells were fixed with 3.7% PFA for 15 minutes at room temperature in the dark. Cells were then washed with PBS, pelleted, and then permeabilized with 0.5% Triton X-100 for 15 minutes at room temperature in the dark. After permeabilization, Click-iT reaction mix was added to each sample proceeded by incubation for 30 minutes at room temperature with rotation in the dark. Cells were then washed with Click-iT Reaction Rinse Buffer (Component F), pelleted, washed once with PBS, and then pelleted again. Cells were resuspended in N3 neuronal media containing DAPI (100×) and then FACS sorted.

EdU Incorporation for FACS Sorting, One day after addition of N3 media (and always omitting glia), EdU incorporation assays ware performed according to manufacturer's instructions (Invitrogen, Cat. No: C10424). Cells were incubated with 1 μM EdU for 1 hour at 37° C., washed once with PBS, dissociated with 0.25% Trypsin-EDTA (Genesee Scientific), resuspended, and spun down. Cells were fixed in 100 uL Click-iT fixative (Component D) and incubated for 15 minutes at room temperature in the dark. Cells were washed with 1% BSA in PBS, pelleted, and resuspended in 100 uL of 1× Click-iT saponin-based permeabilization and wash reagent (Component E) for 15 minutes at room temperature in the dark. Cells were then incubated with Click-iT reaction cocktail for 30 minutes at room temperature in the dark with shaking. Cells were washed with 1× Click-iT saponin based permeabilization and wash reagent (Component E) and then pelleted. Cells were resuspended in N3 neuronal media or PBS containing DAPI (100×) for FACS sorting.

Flow cytometry and FACS analysis. Cells were harvested as previously described for each cell type with trypsin processing for MEFs and 4 dpi samples and DNaseV Papain (Worthington Biochemical) processing for 8 dpi and iMN samples. Sorting of cells for analysis or collection was performed on an Aria I or Aria II (BD). Live single cells were identified by SSC and FSC gating and as DAPI negative. For fixed cells processed for CFSE-EU assays, cells were identified by SSC and FSC gating and DAPI staining was used to identify positive stained cells. Nonfluorescent controls ware included to identify fluorescent populations. For multiple fluorophore experiments, single-labeled cell populations were included to allow proper compensation (e.g., EU-only, EdU-only, CFSE-only controls, primary antibody-only controls, non-labeled cells for CFSE-EU/EdU assays). Sample compensation was performed prior to other analyses. For all CFSE-EU assays, fast cycling cells were determined by gating the dimmest 15% of cells in 6F conditions at 4 dpi. Cells with lower CFSE intensity were gated as fast-cycling. From the fast-cycling population of cells, hypertranscribing cells were identified es the top 50% of the SF only conditions.

Alpha-amanitin treatment for FACS and conversion. Converting cultures were treated with complete N3 media supplemented with water control or α-amanitin (1 μg/mL) at 3 dpi and transcription rate was measured by flow cytometry at 4 dpi using EU incorporation. For iMN conversion, cultures were treated complete N3 media supplemented with water control or α-amanitin (1 μg/mL) from 3-7 dpi, at which point cultures were maintained in complete N3 without water control or a-amanitin until 14-17 dpi.

Aphidicolin, camptothecin, doxorubicin treatment for FACS and conversion. Converting cultures ware treated with complete N3 media supplemented with DMSO control, aphidicolin (1 μM) or doxorubicin (0.25 μM) at 3 dpi for 18 hours. Transcription rate was measured by flow cytometry using EU incorporation or DNA synthesis rate was measured by flow cytometry using Ed U incorporation at 4 dpi. For iMN conversion, cultures were treated with complete N3 media supplemented with DMSO control, aphidicolin (1 μM), camptothecin (1 μM), or doxorubicin (0.25 μM) at 3 dpi for 18 hours, at which point cultures were maintained in complete N3 media without DMSO or small molecules until 14-17 dpi.

Quantification of anaphase-telophase chromatin bridges, micronuclei. For quantification of anaphase-telophase micronuclei or bridges, converting cultures grown on plastic coverslips were fixed with 4% paraformaldehyde at 2 or 4 dpi, respectively. Cells were then stained with DAPI (1:1000) for 10 minutes at room temperature in the dark. After mounting onto glass slides using lmmuMount (Thermo Scientific), cells were acquired on the Zeiss LSM 800 confocal microscope using a 40× objective. Anaphase-telophases with chromatin bridges or micronuclei ware identified based on their DAPI profile as has been previously reported (Slattery et al., 2012; Broderick et al., 2015; Dykhuizen et al., 2013; Kotsantis et al., 2016). Anaphase-telophase cells with one or more non-integrated DNA fragments were determined as having micronuclei. Anaphase-telophase cells with one or more DNA strands between the separating/separated daughter cells were determined as having a bridge. The number of anaphase-telophase mitotic cells with chromatin bridges or micronuclei over all anaphase-telophases was recorded.

Quantification of multipolar neurons. Converted iMN cultures were imaged using the Molecular Devices Image Express at 14 dpi for mouse or at 35 dpi for human and manually quantified using Fiji. Cells expressing the proper marker(s), neuronal morphology, and at least 3 or more neurite processes were included in the quantification of percent multipolar neurons.

RNA Polymerase II+CFSE+EdU labeling for FACS analysis. For CFSE labeling, one day after retroviral infection, fibroblasts were labeled with CellTrace CFSE Cell Proliferation Kit Far Red (Invitrogen, Cat. No: C34572) at a final concentration of 10 μM as described above. For EdU labeling, cells were then incubated with EdU one day after addition of N3 media (without addition of glial cultures) also as described above. After a 30-minute incubation with Click-iT reaction mixture (using Alexa Fluor 594) followed by the wash with 1× Click-iT saponin based permeabilization and wash reagent (Component E), cells were then incubated in 3% FBS in PBS block solution for 30 minutes at room temperature with shaking. After spinning down and resuspending, cells were then incubated with primary antibody (1:200 dilution in 3% block solution) for 45 minutes at room temperature with rotation. Cells were washed with block solution, spun down, and incubated in secondary antibody (1:200 dilution of Alexa Fluor 488 in 3% block solution) for 30 minutes at room temperature in the dark with rotation. Cells were then washed in block solution, spun down, and resuspended in 150-200 μL PBS containing DAPI (100×) prior to FACS sorting and analysis. The following primary antibody was used: rabbit anti-RNA polymerase II CTD repeat YSPTSPS (phospho S2) (Abcam Cat No: ab5095). The following secondary antibody was used: donkey anti-rabbit lgG highly cross-adsorbed secondary antibody, Alexa Fluor 488 (Thermo Fisher Cat. No: A-21206).

Genomic analysis of viral Integrations. To analyze integration of viral constructs into cells during reprogramming, three replicates of 40,000 cells were collected at 4 dpi by trypsinization. To gather cells based on Isl1-GFP expression, populations were collected via FACS for high and low Isl1-GFP as well as gated for CFSE intensity (e.g., CFSE-low for hyperproliferative populations, CFSE-High for slowly dividing cells). Following isolation, cells were pelleted and responded in Direct lysis Buffer (Qiagen) with 1 mg/mL Proteinase K (Qiagen) and processed per manufacturer's instructions. Briefly, cell solutions were incubated at 55° C. for 45 minutes, followed by 85° C. for 1 hour to inactivate Proteinase K. Cell extracts were diluted 1:3 in water. Relative number of integrations were analyzed by qPCR with iTaq Universal SYBR Green Supermix (Biorad) and primers specific for:

the native MALAT1 genomic region primers:
MALAT1-FWD:
(SEQ ID NO: 9)
GGTTTCTCTCTCCCCTCCCT,
MALAT1-REV:
(SEQ ID NO: 10)
TTCGCATACGTGTGTCTGCT,
Isl1-GFP transgene primers:
Isl1-GFP-FWD:
(SEQ ID NO: 11)
AACAGCATGGTAGCCAGTCC,
Isl1-GFP-REV:
(SEQ ID NO: 12)
GCTGAACTTGTGGCCGTTTA,
and
Ngn2-F2A-Isl1 transgene primers:
Ngn2-F2A-Isl1-FWD:
(SEQ ID NO: 13)
GAGAAGCATCGTTATGCGCC,
Ngn2-F2A-Isl1-REV:
(SEQ ID NO: 14)
TCCCATTGGACCTGGATTGC.

Relative integrations were determined by calculating each samples delta CT for the transgenes relative to the native MALAT1 region and calculating 2 raised to the negative delta CT.

Single cell qPCR. Single iMNs of different morphologies were identified and isolated using an inverted microscope equipped with micromanipulator and micropipette. Cells were collected directly into 54 of CellsDirect 2× Buffer (Cells Direct One-step qRT-PCR kit, Thermo). Cells were processed using the manufacturer's protocol for reverse transcription (RT) and specific target amplification (STA). cDNA was synthesized and pre-amplified from single-cell lysate. Single-cell qPCR was performed using the Fluidigm BioMark HD system on amplified cDNA templates, with primer and SsoFast EvaGreen supermix. Primers were validated in-house to yield efficient PCR amplification. A matrix of C_{T}s and quality metrics was generated and extracted for each cell. Cells and genes were excluded for low-quality scores. In all, expression across 17 genes for fibroblast and neuronal markers was performed for 25 fibroblast-like cells and 36 neuronal cells. A profile of expression was generated for each cell using delta C_{T}s normalized across total expression of the panel of genes. A heatmap was generated to visualize the profile of expression across the different gene sets and morphologies.

Single cell RNA-sequencing. Cells were harvested at different points in conversion. Specific populations were identified and collected via FACS and all cells were sorted to obtain viable single cell suspensions. Fast-cycling cells were identified by low CFSE intensity at 4 dpi. Hb9::GFP+ at 8 dpi and 14 dpi cells were identified relative to Hb9::GFP negative control. Cell suspensions were loaded into a chip and processed with the Chromium Single Cell Controller (10× Genomics). To generate single-cell gel beads in emulsion (GEMs), individual populations were assigned individual libraries using Single Cell 3′ library and Gel Bead Kit V2 (10× Genomics, 120237). For each population, the target population size was between 1000-1500 cells. Cell suspensions were calibrated to capture the target number of cells. Fewer cells were captured at 8 dpi due to limited Hb9::GFP+ cells in 6F condition. RNA from lysed cells was barcoded through reverse transcription in individual GEMs. Barcoded cDNAs were pooled and cleanup by using DynaBeads® MyOne Silane Beads (Invitrogen, 370020). Single-cell RNA-seq libraries were prepared using Single Cell 3′ library Gel Bead Kit V2 (10× Genomics, 120237). Sequencing was performed with using multiple NextSeq 500/550 High Output Kit v2 on an Illumina NextSeq with pair end 150 bp (PE150). On average, sequencing generated 100-200K reads per cell on average over the libraries.

Single Cell RNA-Seq Analysis:

Cluster analysis via Seurat. Analysis of embryonic motor neurons and induced motor neurons from various conditions was performed using Seurat 2.2. Following alignment and processing in CellRanger, variable genes were identified using FindVariableGenes. Clustering was performed using FindClusters based on the number of PCs identified through the PCElbow plot function. Cluster markers were identified for each cluster using the FindMarkers function.

Cluster analysis and pseudo temporal ordering via Monocle. The Cellranger count pipeline (10× Genomics) was used to align and quantify single cell expression for each library. Samples were combined into a single matrix via the aggr pipeline and normalized by read depth across the libraries. scRNaseq datasets were imported into Monocle using CellrangerRkit in R to create a cellDataSet. Data were normalized using estimateSizeFactors. Outliers were removed based on variance using estimateDispersion to remove 108 outlier cells. Clustering was performed using 10,300 genes with high dispersion and mean gene expression >=0.1 on the first 10 PCs. Clusters of varying number were examined and clustering via 3 primary clusters was chosen to capture different populations (e.g., MEFs, converting cells, and iMNs). Pseudotemporal ordering was performed using identified clusters. Pseudotemporal ordering was rooted in the identified iMN endpoint. To generate the pseudotime trajectory corresponding to reprogramming time, pseudotime was reversed to generate trajectory spanning MEFs at t=0 and iMNs at t=30 (end time). All subsequent pseudo time analyses were performed with the resulting cellDataSet.

Bulk RNA-sequencing and analysis. For cultures at 17 dpi, cells were harvested by DNase/papain (Worthington Chemical) treatment to dissociate cells. Cells were washed three times in DMEM-F12 media and resuspended in N3 neuronal media for sorting. Cells in replicates of 50K were collected based on gates set to identify viable, single Hb9::GFP+ cells. Following sorting, cells were spun and resuspended in 100 uL RLT buffer from the RNAeasy micro kit (Qiagen). RNA in RLT and RNA extracted via RNAeasy kit were sequenced by Amaryllis (Emeryville, Calif.) via single-end sequencing to generate 30M reads per sample. Additionally, Fastq files for previously acquired data for MEFs, embryonic motor neurons (embMNs), iPSC-derived MNs, and ES-derived MNs samples in duplicate were acquired and processed with newly generated datasets. Sequencing reads from triplicate or more replicates were trimmed and aligned to mm 10 reference transcriptome with STAR aligner 2.5.3a. Gene counts quantified using annotation model (Partek E/M). Differentially expressed genes were identified using DEseq2 for with genome wide false discovery rate (FDA) of less than 0.05 and log 2 fold change greater than 1. Comparison of MEFs with all MN samples generated 1186 DEGs. Heatmap analysis of MEFs and iMNs from different conditions was generated using this DEG set. Direct comparison of iMNs from 6F and DDRR conditions generated 756 DEGs. Metascape analysis (www.metascape.org) was used to generate GO terms for up and downregulated genes.

Cell number normalized (CNN) RNA-sequencing and analysis. For cultures at 4 dpi, cells were harvest by trypsin treatment to dissociate cells. Cells were washed three times in DMEM-F12 media and resuspended in N3 neuronal media for sorting. Cells in replicates of 50K were collected based on gates set to identify viable, fastcycling cells (e.g., CFSE-lo) or each condition (e.g., SF and DORA). Following sorting, cells were spun and resuspended in 100 uL RLT buffer from the RNAeasy micro kit (Qiagen). To normalize to a standard number of cells, ERCC spike-in mix (ThermoFisher, 1 uL at 1:100 dilution) was added to 50K cells in RLT. RNA in RLT and RNA extracted via RNAeasy kit, libraries were prepared by DNAlink (San Diego, Calif.) using SMARTer Stranded Total RNA-Seq Kit-Pico Input Mammalian (Clontech) and were sequenced using NextSeq 500 Mid-output 75PE (Illumina) to generate 30M reads per sample. Sequencing reads from triplicate or more replicates were trimmed and aligned to mm10 reference transcriptome with STAR aligner 2.5.3a. Gene counts quantified using annotation model (Partek E/M). Samples were aligned to ERCC spike-in reference to quantify total spike-in reads per sample. Sample reads were normalized by spike-in reads to generate cell number normalized reads per sample.

Biotinylated-trimethylpsoralen (bTMP) Immunofluorescence. One day after addition of N3 media (and without addition of glial cultures), cells were treated with 1 μM aphidicolin for 1.5-2 hours in N3 media. For control experiments, MEFs were treated with or without 100 μM bleomycin prior to incubation with psoralen. Then cells were incubated with 0.3 mg/mL EZ-Link Psoralen-PEG3-Biotin (Thermo Cat. No: 29986) for 15 minutes. Cultures were then exposed to 3 kJ m-2 of 365 nM light (Fotodyne UV Transilluminator 3-3000 with 15W bulbs) for 15 minutes at room temperature in the dark followed by 3 washes in PBS. Then cells were fixed with cold 70% ethanol for 30 minutes at 4° C. followed by another 3 washes in PBS. Cells were then incubated with Alexa Fluor 594 Streptavidin (Thermo Cat. No: 532356) for one hour at room temperature in the dark, washed with PBS 3 times, and then stained with Hoescht (1:1000) for 10 minutes at room temperature in the dark. Coverslips were mounted onto glass slides using lmmuMount and imaged using the Zeiss LSM 800 confocal microscope.

Trimethylpsoralen-qPCR.

Cell Harvest and DNA Extraction. One day after addition of N3 media (and without addition of glial cultures), cells were treated with 1 μM aphidicolin for 1.5-2 hours in N3 media. Cells were then trypsinized in 0.25% Trypsin-EDTA, spun down, and resuspended in complete N3 media+1 μM aphidicolin+2 μg/ml trimethylpsoralen (Sigma). 500 μL of control-puro was removed and saved for the no UV crosslinking control. Each 1 mL of the remaining samples were added to individual wells of a 24-well plate and then exposed to 3 kJ m-2 of 365 nM light (Fotodyne UV Transilluminator 3-3000 with 15W bulbs) for 15 minutes at room temperature in the dark. Cells were then re-collected, spun at 1000×g for 5 minutes, washed with 1 mL PBS, and spun down again. Then cells were resuspended in 200 μL PBS and purified using Qiagen DNeasy Blood and Tissue Kit with inclusion of an RNase A digestion (Qiagen Cat. No: 69504). Samples were eluted once in 200 μL followed by a second elution in 100 μL of Buffer AE and eluates were then combined.

Sonication, Quantification, and Exonuclease I Digestion. To achieve fragment sized of 100-500 bp, each sample was sonicated in a Bioruptor for 30 s on/30 s off for 45 minutes on High. To ensure the same amount of DNA was then used for Exonuclease digestion, sample concentrations were quantified with a qPCR reaction. Briefly, samples were heat denatured at 95° C. for 10 minutes, put on ice for 2 minutes, and then spun down briefly. For the qPCR reaction, 2 μL DNA for each sample was used in a 20 μL iTaq Universal SYBR Green Supermix (Biorad Cat. No: 1725125) reaction using primers −500 bp upstream of the TSS for Actb. The qPCR results were used to determine the relative concentrations of each sample, using the least concentrated sample as the reference to adjust all other sample concentrations to. Samples were brought to a total volume of 280 μL after adjustment for DNA concentration and then heat denatured at 95° C. for 10 minutes followed by a 2-minute recovery on ice. Then, 240 μL of each sample was put into a new tube, saving the remaining undigested 40 μL of DNA at 4° C. The 240 μL samples were then heat denatured at 95° C. for 10 minutes, incubated on ice for 2 minutes, and then briefly spun down. To each 240 μL sample, the following was added: 29 μL 10× Exonuclease I buffer+1 μL Exonuclease I and samples were incubated at 37° C. for one hour. Samples were then heat denatured at 95° C. again, put on ice for 2 minutes, spun down, and another 10 μL of Exonuclease I was added. After another 1-hour incubation at 37° C., samples were heated at 95° C. for 10 minutes and put on ice for 2 minutes to stop the exonuclease reaction.

TMP-qPCR. The non-exonuclease digested samples were diluted 1:8 in Milli-Q water to a total volume of 320 μL A qPCR reaction was then performed on both exonuclease digested and non-exonuclease digested samples with the upstream primers (−500 bp from TSS) for several genes. Inclusion of non-exonuclease digested samples were used to normalize input levels for each exonuclease treated sample. Each biological sample was run in technical triplicate using 4 μL DNA per well in a 20 μL iTaq Universal SYBR Green Supermix reaction using the ViiA 7 Software. The following primers were used for qPCR quantification:

Acfb
(SEQ ID NO: 15)
5′-GTCTCGGTTACTAGGCCTGC-3′
(SEQ ID NO: 16)
5′-ATCCACGTGACATCCACACC-3′
Gapdh
(SEQ ID NO: 17)
5′-GGTGAGATCAGTGAGGGGAG-3′
(SEQ ID NO: 18)
5′-CAAGAGGCTAGGGGCTTCC-3′
Sod1
(SEQ ID NO: 19)
5′-TCCGCATTTCCAGACACAGT-3′
(SEQ ID NO: 20)
5′-GAGCGGGGAAAGTCGCTATT-3′

Live imaging. Live imaging was carried out using a Nikon Biostation CT.

Quantification and Statistical Analysis. Sample numbers and experimental repeats are indicated in figure legends. Unless otherwise stated, data presented as mean±SEM of at least three biological replicates. Significance determined by one-way ANOVA for multiple comparisons while an unpaired t test was used when comparing two datasets. If a dataset was non-normally distributed according to the D'Agostino & Pearson omnibus normality test, Kruskai-Wallis or Mann-Whitney testing was used for multiple comparisons or when comparing two datasets, respectively. Significance summary: p>0.05 (ns), *p≤0.05, **p≤0.01, ***p≤0.001, and -p≤0.0001.

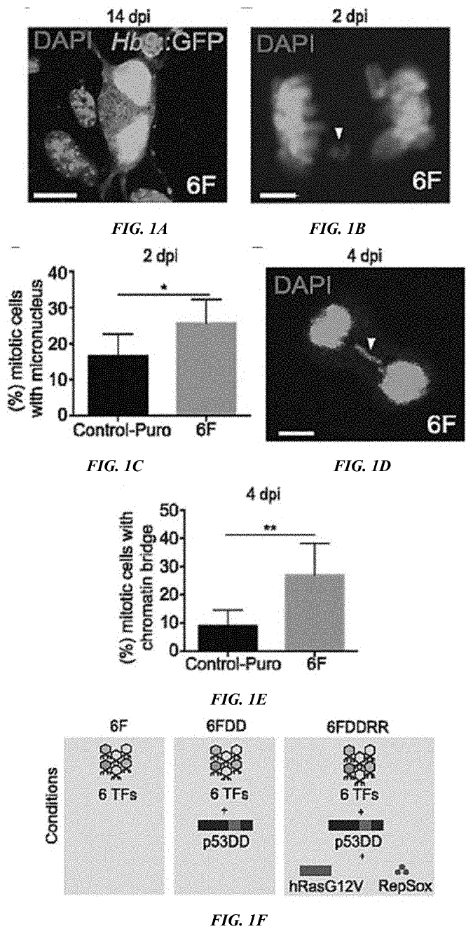

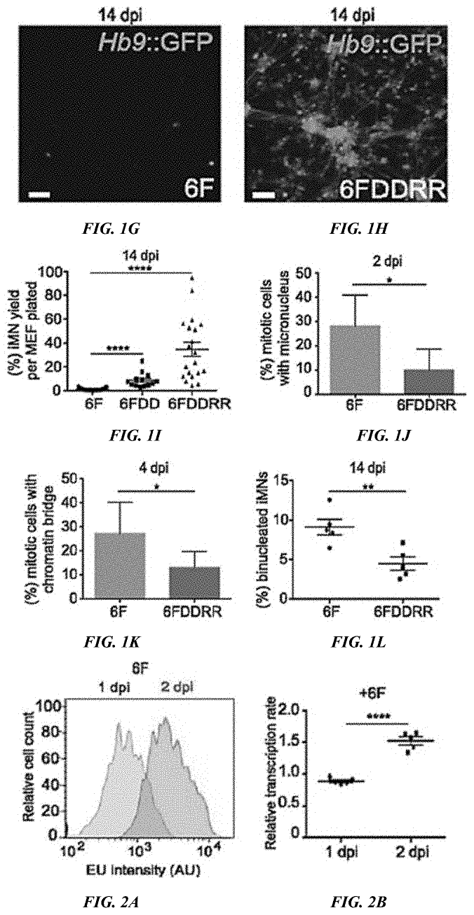

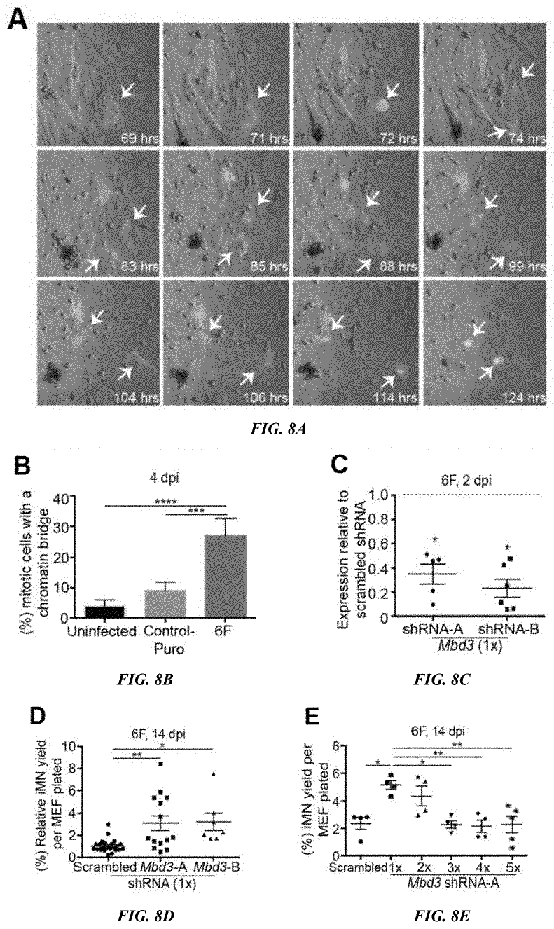

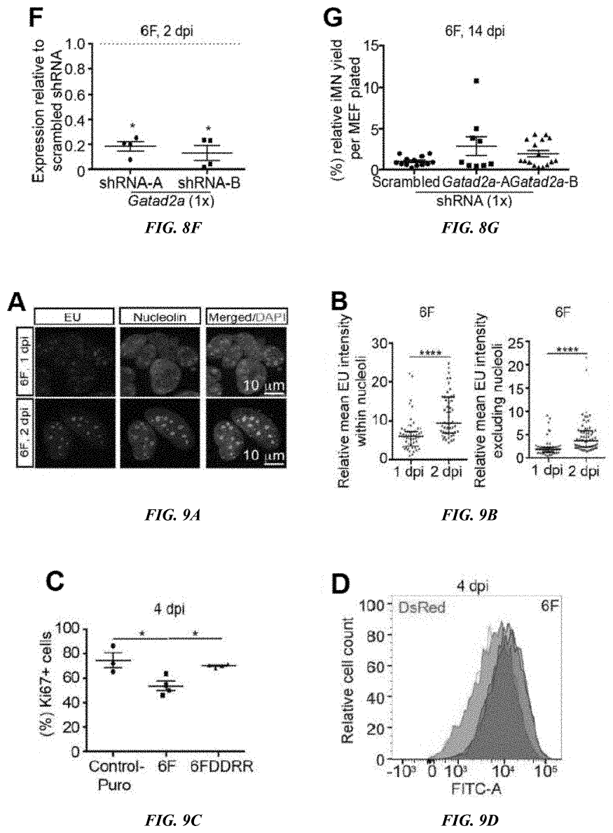

Transcription Factor Overexpression Induces Genomic Stress. The motor neuron lineage was focused on because it is a well-defined neuronal subtype with established markers. Utilizing mouse embryonic fibroblasts (MEFs) isolated from Hb9::GFP transgenic mice, motor neurons (iMNs) were generated by viral overexpression of six transcription factors (Ascl1, Bm2, Mytl1, Ngn2, Isl1, and hx3 [6F]). A large number of binucleated iMNs (˜10%; FIG. 1A) were observed, suggesting cell division and incomplete cytokinesis during reprogramming. Using longitudinal tracking from 1 to 8 days post-infection (dpi), it was found that cells activated Hb9::GFP following division, definitively indicating that reprogramming cells do divide (see FIG. 8A).

Impaired DNA replication can cause failed cytokinesis, chromatin bridges between separating nuclei, and micronuclei as the chromatin bridges resolve. Transduction with the iMN factors, but not a puromycin resistance gene (Control-Puro), induced DAPI+ micronuclei and chromatin bridges in ˜30% of the mitotic anaphase-telophase cells at 2 and 4 dpi, respectively (see FIGS. 1B-E and FIG. 8B). Thus, cell division occurs during iMN reprogramming and transcription factor overexpression induces DNA replication stress.

Identification of a Genetic and Chemical Cocktail that Massively Increased Reprogramming. To identify factors that promote lineage conversion into somatic cell types, small molecule kinase inhibitors, epigenetic modifiers, and oncogenes were screened for the ability to increase the efficiency of MEF-to-IMN reprogramming. Suppression of Gatad2a-Mbd3/NuRD enables deterministic iPSC reprogramming. In partial agreement, Mbd3 suppression modestly increased iMN reprogramming (see FIG. 8C-E). However, unlike in iPSC studies, Gatad2a suppression did not increase IMN reprogramming (see FIG. 8C-E). Thus, Gatad2a-Mbd3/NuRD does not regulate iMN reprogramming as strongly as it regulates iPSC reprogramming.

A combination of RepSox, a transforming growth factor β(TGF-β) inhibitor, a Ras mutant (hRasG12V), and p53DD (DD), a p53 mutant lacking a DNA-binding domain (see FIG. 1F), increased iMN reprogramming by 100-fold (see FIG. 1G-I). In reprogramming cultures, DD, RepSox, and hRasG12V significantly reduced micronuclei, chromatin bridges, and binucleated iMNs (see FIG. 1J-L). This suggests a strong correlation between reducing genomic stress and increased iMN formation.

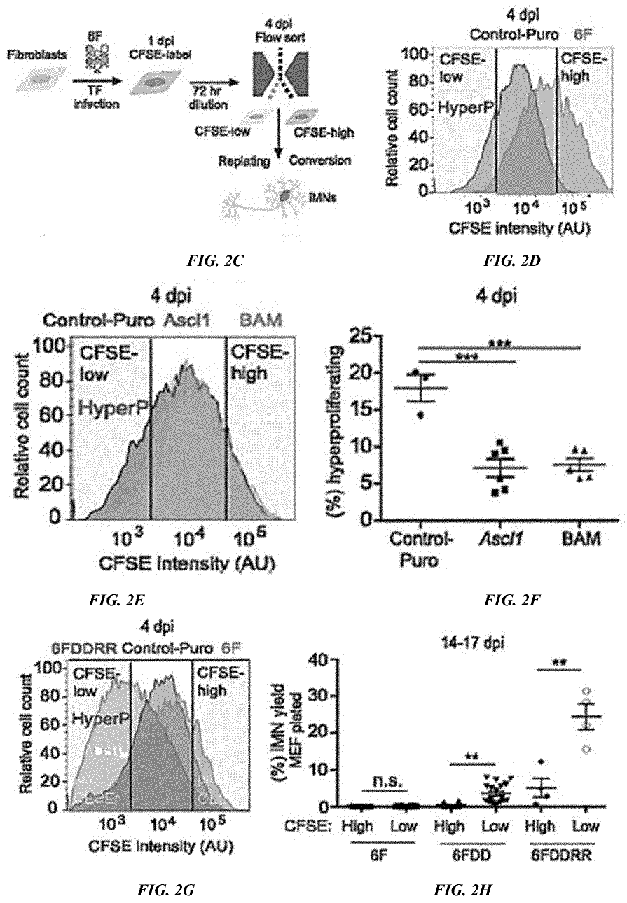

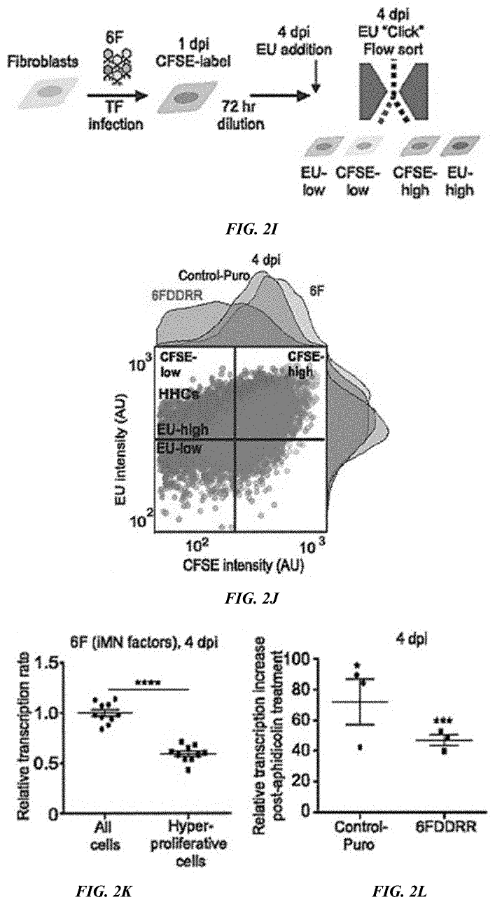

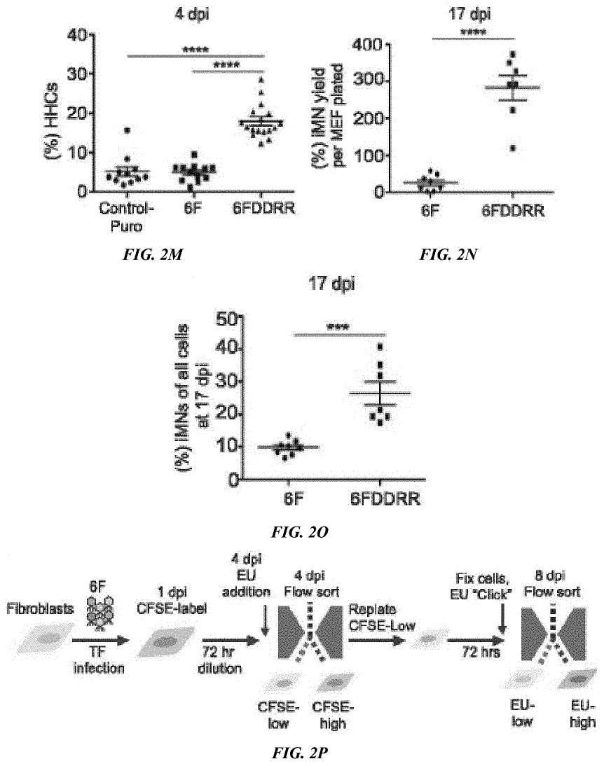

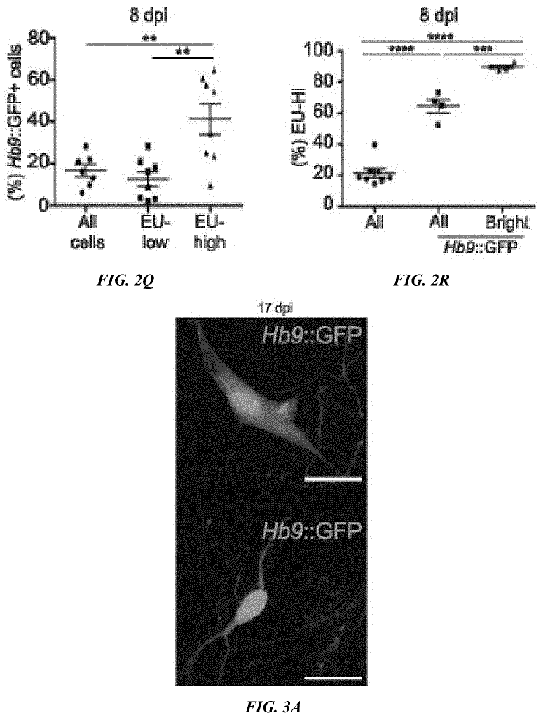

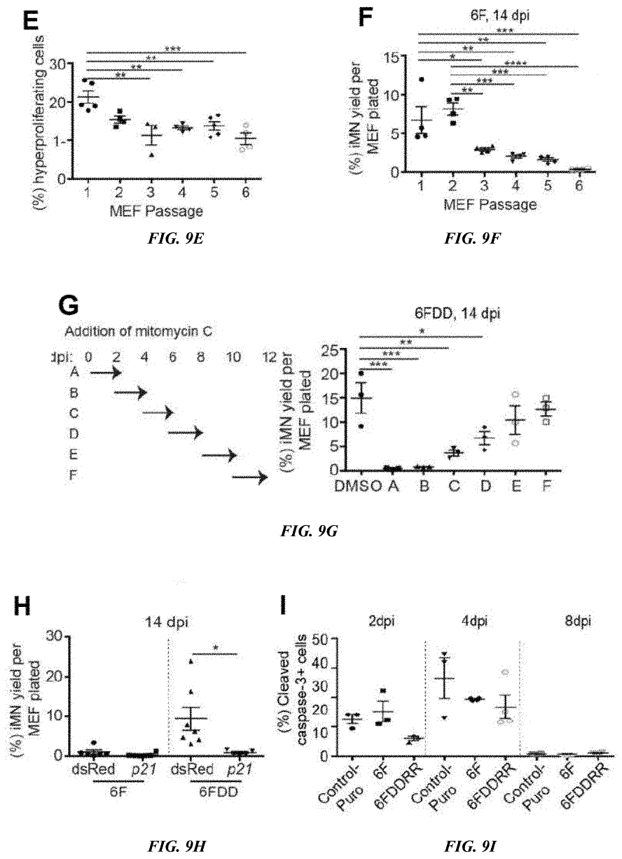

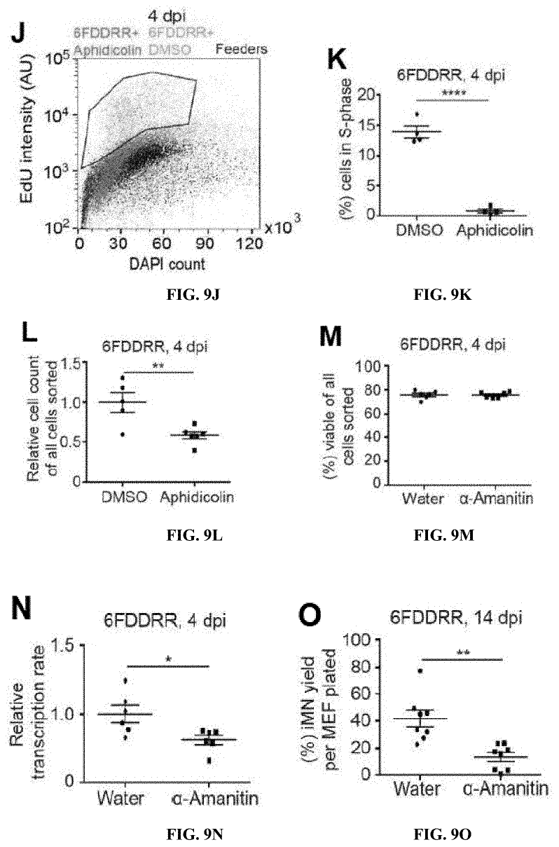

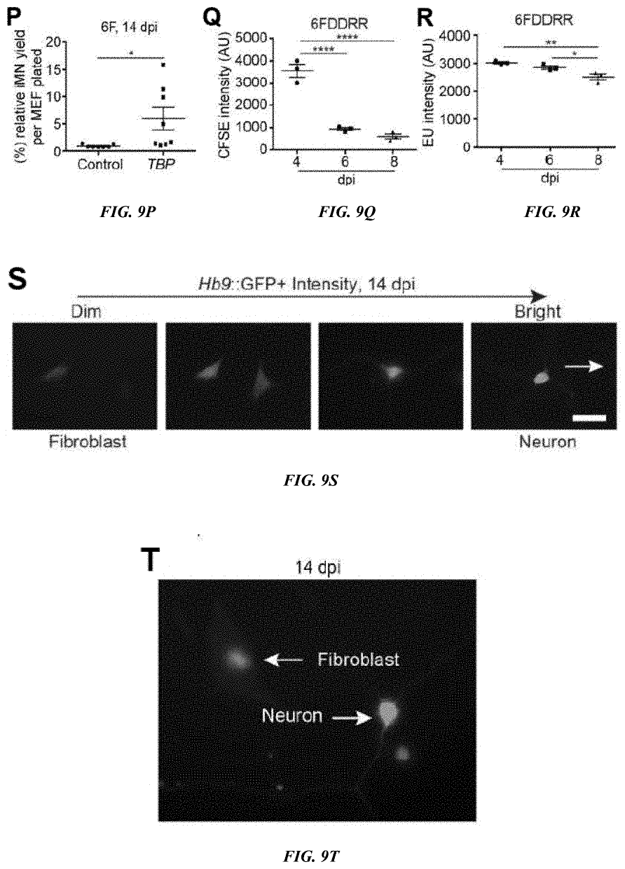

Hypertranscription and Hyperproliferation Drive Neuronal Reprogramming. Transcription and DNA replication antagonize each other by increasing torsional strain and steric interference on genomic DNA. Measuring 5-ethynyl uridine (EU) incorporation by fluorescence-activated cell sorting (FACS) at 2 dpi (see FIG. 9A) revealed that 6F transduction induced a significant increase in mean EU intensity in MEFs (see FIG. 2A). The relative transcription rate was defined as the mean EU incorporation of SF-transduced MEFs relative to non-transduced MEFs on the same day. The transcription rate in 6F transduced MEFs increased 50% from 1 to 2 dpi (see FIG. 2B). Nuclear EU signal in 6F MEFs increased within and outside of nucleoli at 2 dpi compared to 1 dpi (see FIG. 9A-B), suggesting that both RNA-polymerase-I and II-dependent transcription are elevated. Because this reflected an increased overall transcription rate in MEFs, this state was termed “hypertranscription.”

Next was evaluated the impact of 6F transduction on cell proliferation. Cell proliferation was measured by labeling MEFs with the stable dye CFSE (carboxyfluorescein succinimidyl ester) 24 h after transduction and flow sorting 72 h later (see FIG. 2C). Similar to published studies, “hyperproliferation” or “fast-cycling” cells was defined as those showing a two-fold increase in division rate (an eight-fold decrease in CFSE intensity) compared to control MEFs. 6F transduction reduced the percentage of hyperproliferating cells ten-fold (see FIG. 2D). Staining for Ki67 at 4 dpi confirmed that 6F reduced the proliferative population and DDRR restored it (see FIG. 9C). OsRed retrovirus did not reduce proliferation, suggesting the effect was specific to transcription factors (see FIG. 9D). Ascl1 alone or Bm2, Ascl1, and Myt11 (BAM) also significantly reduced hyperproliferative cells (see FIG. 2E-F). Thus, transcription factor overexpression reduces cell proliferation.

DDRR greatly increased the number of hyperproliferating cells during iMN reprogramming (see FIG. 2G). Reprogramming cultures were labeled with CFSE at 1 dpi, prospectively isolated hyperproliferative cells by FACS 72 h after CFSE labeling (at 4 dpi), and measured their ability to form iMNs by 14-17 dpi (see FIG. 2C). In 6F, 6F+DD (6FDD), and 6FDDRR conditions, cells that hyperproliferated from 1 to 4 dpi formed iMNs at higher rates (see FIG. 2H). Reducing cell division by MEF passaging, mitomycin C treatment, or p21 overexpression impaired iMN conversion (see FIG. 9E-H). Mitomycin C treatment at different time points indicated that cell division early in conversion promotes reprogramming (see FIG. 9G). DDRR did not reduce apoptosis during reprogramming (see FIG. 9I). Importantly, hyperproliferative cells only reprogrammed with substantially greater efficiency in the 6FDD and 6FDDRR conditions, suggesting that DDRR provided hyperproliferative cells with additional properties that enabled efficient reprogramming (see FIG. 2H).

Next were measured cellular proliferation and transcription rates during reprogramming with DDRR (see FIG. 2I). Hypertranscribing cells were defined as cells in the top half of EU intensity within the hyperproliferating population in the 6F condition (see FIG. 2J). Hyperproliferating cells displayed significantly reduced transcription levels when transduced with the six iMN factors (see FIG. 2J-K). Aphidicolin dramatically reduced the percent of cells in S phase as measured by EdU incorporation at 4 dpi while only slightly reducing cell count (see FIGS. 9J-L), and this increased the transcription rates in Control-Pure and 6FDDRR cells (see FIG. 2L). Thus, transcription and DNA synthesis oppose each other during reprogramming.

DDRR increased the transcription rate of SF-infected hyperproliferative cells, resulting in a larger population of HHCs (see FIG. 2J-M; hypertranscribing and hyperproliferating cells defined as above in FIGS. 2J and 2D, respectively). a-amanitin treatment at 4 dpi to reduce the average transcription rate in 6FDDRR cells did not reduce viability but significantly impaired reprogramming (see FIG. 9M-O). Conversely, overexpressing TATA-binding protein (TBP), which increases transcription in fibroblasts, significantly increased iMN reprogramming in the 6F condition (see FIG. 9P). Thus, there is a strong correlation between hypertranscription, hyperproliferation, and the rate of iMN conversion, and DDRR increases HHCs after 6F infection.

Given the high density of Hb9::GFP+ cells in 6FDDRR conditions (see FIG. 1G-H), quantification of reprogramming efficiency was improved by generating 6F or 6FDDRR iMNs without primary glia and exhaustively quantifying cell number by flow cytometry. Although 6F resulted in less than 10 IMNs per 100 MEFs plated, 6FDDRR yielded ˜300 iMNs (see FIG. 2N). Without DDRR, 90% of cells failed to activate Hb9::GFP. With DDRR, 30% of cells activated Hb9::GFP (see FIG. 2O). Because HHCs represent 20%-30% of 6FDDRR cells (see FIG. 2M) and comprise the majority of reprogrammable cells (see FIG. 2P-2R), 30% of MEFs activating Hb9::GFP represent near-deterministic reprogramming of the HHC population. Thus, DDRR boosts reprogramming by increasing the number of cells capable of maintaining hypertranscription and hyperproliferation early in conversion.

To test whether HHCs identified at 4 dpi possess greater reprogramming potential relative to hyperproliferative cells with lower transcription rates, HHCs and non-HHCs were prosectively isolated (see FIG. 2P). Hb9::GFP MEFs were CFSE-labeled at 1 dpi, pulse-labeled cells with EU at 4 dpi prior to isolating CFSE-low, hyperproliferative cells by FACS, and re-plated these cells from 4 to 8 dpi. From 4 to 8 dpi, CFSE dimmed 10-fold and EU signal only dropped 10% (see FIG. 9Q-R). Using FACS at 8 dpi, HHCs were identified by high EU levels and analyzed them for Hb9::GFP expression (see FIG. 2P). Cells with EU intensity in the top quartile of hyperproliferative cells were used to stringently examine hypertranscribing cells.

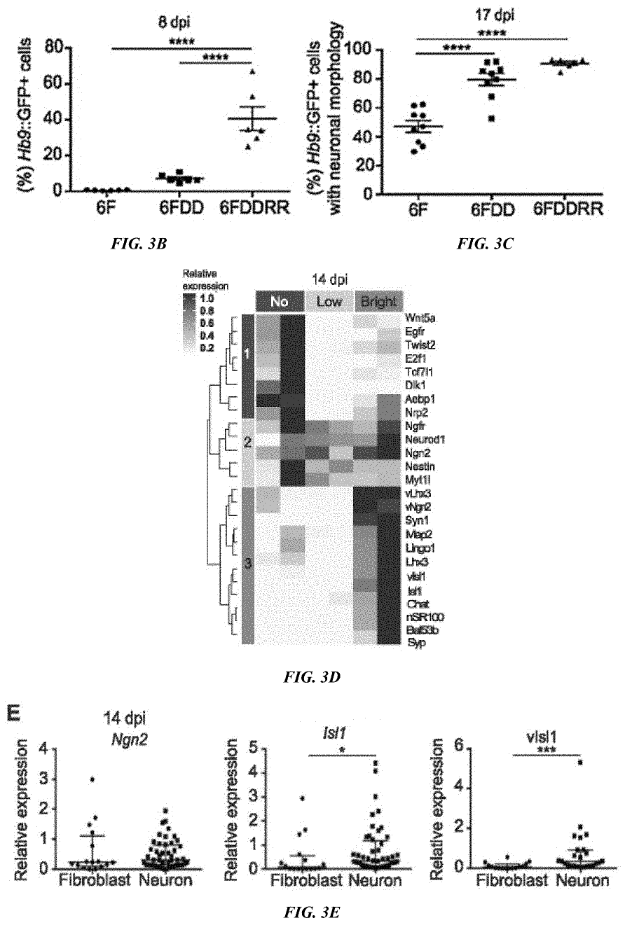

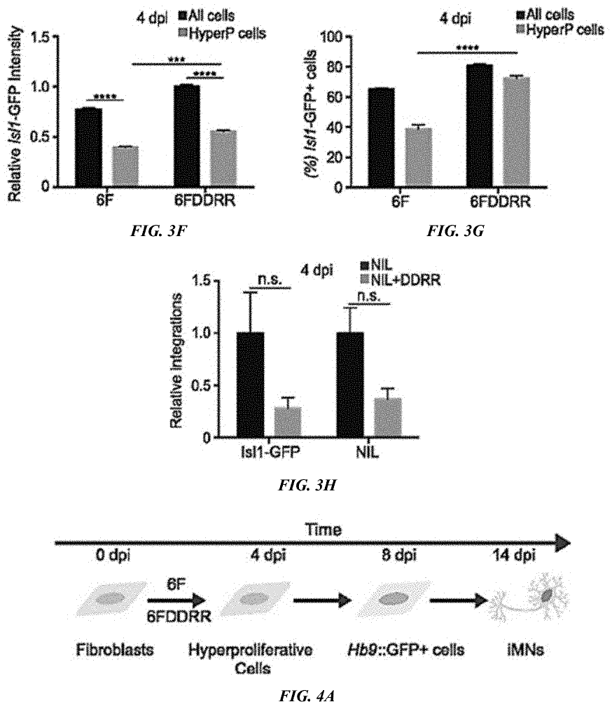

Over 40% of HHCs expressed Hb9::GFP at 8 dpi, although only 13% of non-hypertranscribing cells were Hb9::GFP+ (see FIG. 2O). Thus, HHCs were 3 times more likely to activate Hb9::GFP relative to hyperproliferative but non-hypertranscribing cells. 90% of bright Hb9::GFP+ cells, which display better neuronal morphology and gene expression than low Hb9::GFP+ cells (see FIGS. 9S-T, and FIG. 3D), had an EU intensity in the top quartile of all cells. Thus, of the Hb9::GFP+ cells that advanced to the terminal iMN stage, most originated from HHCs (see FIG. 2R). These prospective isolation studies indicate that HHCs possess significantly greater reprogramming potential than non-HHCs, and the inability of most cells to sustain hypertranscription and hyperproliferation early in conversion limits reprogramming to rare cells. Increasing the population of cells capable of mediating both processes improves reprogramming to near deterministic rates.

Sustained Transgene Expression Differentiates Complete from Partial Reprogramming. Previous research showed that components of the fibroblast GRN remain active within induced neurons (iNs). Potentially, mechanisms limiting reprogramming may arrest cells at intermediate states, leading to heterogeneous cultures composed of fully and partially neuronal cells.

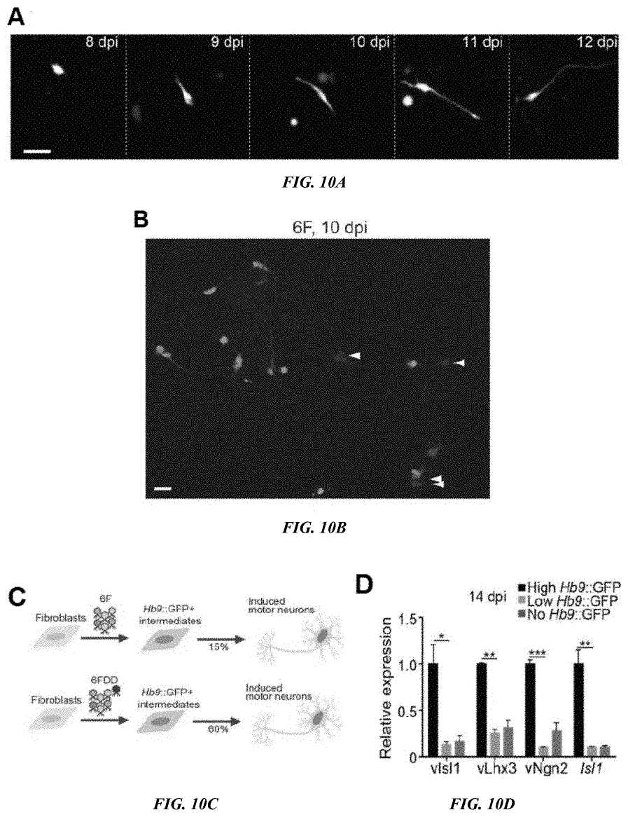

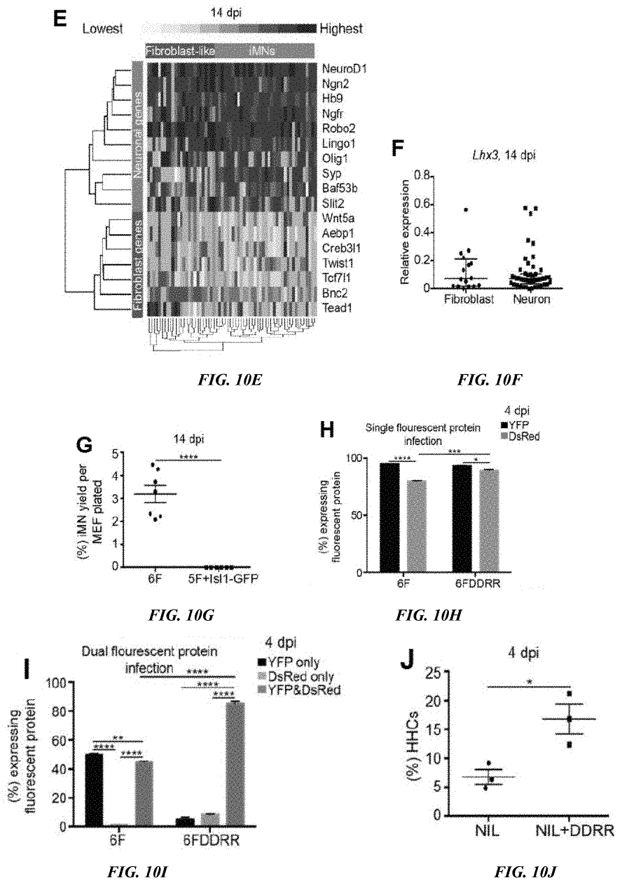

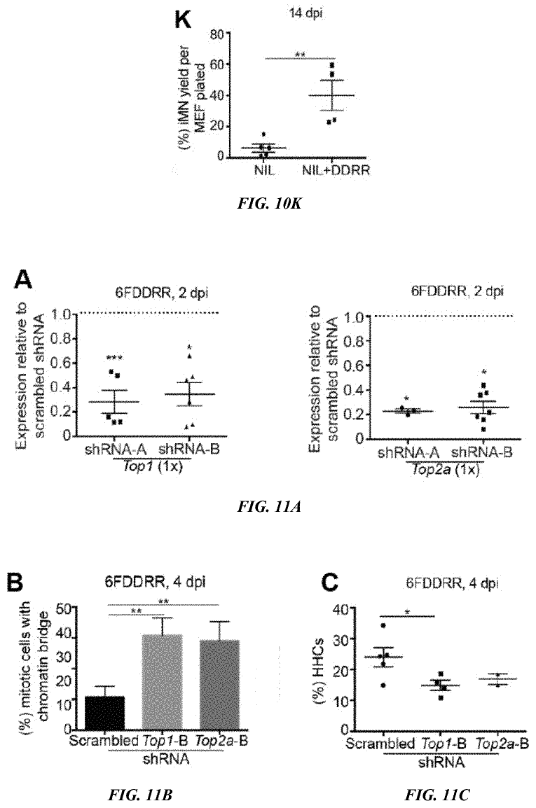

Using live imaging, a post-mitotic intermediate state characterized by Hb9::GFP reporter activation and retention of a fibroblast morphology was identified (see FIG. 3A, top panel). This state frequently preceded Hb9::GFP+ iMN formation (see FIG. 10A), and 50% of Hb9::GFP+ cells remained trapped in this state with 6F alone (see FIG. 10B). Longitudinal tracking showed that, in the presence of DD, Hb9::GFP+ intermediates were four times more likely to fully convert into iMNs (see FIG. 10C; n=65-80 cells in 6F and n=1,200-1,400 in 6FDD). FACS purification of Hb9::GFP+ cells at 8 dpi showed that less than 1% of 6F cells activated Hb9::GFP, and 8% and 40% of 6FDD and 6FDDRR cells activated Hb9::GFP, respectively (see FIG. 3B). Additionally, although 50% of 6F Hb9::GFP+ cells remained trapped in the fibroblastic intermediate state, 90% of 6FDDRR Hb9::GFP+ cells became iMNs by 17 dpi (see FIG. 3C).

To identify transcriptional patterns that differentiate successful from unsuccessful reprogramming, cells were collected at 14 dpi, flow sorted based on Hb9::GFP+ into No, Low, and Bright Hb9::GFP populations, and performed qRT-PCR analysis. Cells lacking Hb9::GFP (No, top, gray) expressed high levels of a cluster enriched with fibroblast genes (cluster 1, left, gray; see FIG. 3D). Cluster 3 (left, bright green) genes were enriched with transgenes and neuronal markers and highly expressed in the Bright Hb9::GFP population (Bright, top, bright green; FIG. 3D). Hb9::GFP Bright cells were more neuronal and showed sustained transgene expression compared to No and Low Hb9::GFP cells (see FIG. 3D and FIG. 10D), suggesting that the ability to sustain exogenous reprogramming transcription factor expression until 14 dpi is critical for reaching the Bright Hb9::GFP+ iMN state.

Single-cell qRT-PCR showed that iMNs (see FIG. 3A, bottom) displayed increased expression of neuronal markers relative to Hb9::GFP+ fibroblast-like intermediates (see FIG. 10E). Hb9::GFP+ fibroblasts did not show substantially more fibroblast gene expression than Hb9::GFP+ neurons, suggesting that activating neuronal gene expression rather than suppressing fibroblast expression was the limiting step in the Hb9::GFP+ intermediate stage (see FIG. 10E). In particular, high expression of transgenic and endogenous Isl1 differentiated neuronal from fibroblast morphologies (see FIG. 3E and FIG. 10F).

To examine transgene expression during reprogramming, an Isl1-GFP fusion construct was constructed. Isl1-GFP was insufficient to replace Isl1 in reprogramming, suggesting the fusion impacted Isl1 function (see FIG. 10G). Although Isl1-GFP intensity dropped in hyperproliferative cells in both 6F and 6FDDRR conditions (see FIG. 3F), DDRR doubled the percentage of hyperproliferative cells with detectable Isl1-GFP expression, suggesting DDRR could sustain transgene activation in hyperproliferative cells (see FIG. 3G). To evaluate how multiple virus expression varied in 6F and 6FDDRR, YFP- and DsRed-labeled viruses were used (see FIG. 10H-I). Although individual viruses showed high expression efficiency in 6F and 6FDDRR after single-virus infections (80%-90%; see FIG. 10H), the percentage of cells exhibiting detectable expression of both fluorescent proteins upon double infection was significantly higher in 6FDDRR than 6F (see FIG. 10I).

To measure transgenic integrations in a relevant context but eliminate the complexity of 6 individual transcription factors, a polycistronic cassette of Ngn2, Isl1, and Lhx3 (NIL) was constructed. These factors reprogram embryonic stem cells (ESCs) to motor neurons. NIL is sufficient to mediate reprogramming, and DDRR increased HHCs and reprogramming in this system (see FIG. 10J-K). At 4 dpi, NIL+DDRR did not have more integrations of the NIL cassette than NIL-alone cells (see FIG. 10H). Similarly, NIL+DDRR cells transduced with Isl1-GFP from the previous experiments did not contain more Isl1-GFP transgenes than NIL calls (see FIG. 3H). Thus, DDRR does not enable higher transgene expression by increasing the number of transgene integrations. Instead, DDRR enables higher levels of transgene expression in hyperproliferative cells, leading to efficient activation of Hb9::GFP and transition of partially reprogrammed intermediates to the neuronal state. Therefore, transgene expression levels in proliferating cells, rather than viral transduction rates, limit reprogramming.

Topoisomerase Enable Simultaneous Hypertranscription and Hyperproliferation in HHCs. To determine how DDRR enables combined hypertranscription and hyperproliferation, RNA sequencing (RNAseq) was performed on single cells on a successful reprogramming trajectory by profiling hyperproliferative cells at 4 dpi (CFSE-Iow) and Hb9::GFP+ cells at 8 and 14 dpi (see FIG. 4A). 6F and 6FDDRR iMNs were similar to each other relative to MEFs and reprogramming cells, suggesting cells take similar trajectories to the iMN state in either condition (see FIG. 4A-B). At 4 dpi, 6F and 6FDDRR cells mapped to similar locations (see FIG. 4B). At 8 dpi, pseudotima analysis indicated more BFDDRR calls were proximal to the iMN state than 6F cells (see FIG. 4C; note color scheme is consistent among FIG. 4C and FIGS. 4E-4H are distinct from that in FIG. 4B). Thus, 6F and 6FDDRR cells traverse through a conserved trajectory, but DDRR increases reprogramming speed and efficiency.

Next was examined the different single-cell states to identify transcriptional programs enabling combined hypertranscription and hyperproliferation (see FIG. 4D-4H). As expected, converting cells decreased collagen gene expression during transit to iMNs and increased Map2, a marker of post-mitotic neurons (see FIG. 4F). Monocle 2 clustered cells based on differentially expressed genes and aligned cells along a reprogramming trajectory. Most state 1 cells remained close to the starting fibroblasts, although some 6F iMN cells clustered into state 1 most likely based on sustained Col1a1 expression (see FIG. 4E). In contrast, state 2 cells possessed a proliferative signature and high expression of Mki67 (see FIG. 4F-G). Because about 80% of MEFs were Ki67+ by immunostaining (see FIG. 10B), the low level of Mki67 in state 1 is due to the limited sensitivity of single-cell RNA-seq and indicates that Mki67 increases in state 2 above levels observed in proliferating MEFs. State 2 was also enriched in unique molecular identifiers (UMis), a proxy of total mRNAs (see FIG. 4O and FIG. 4G), signifying a putative HHC population.

State 2 cells showed increased expression of two topoisomerases (see FIG. 4H). Top1 expression increased at early stages and was sustained throughout reprogramming, and Top2a peaked as cells transitioned from fibroblasts (high Col1a1; low Map2) to iMNs Qow Col1a1; high Map2; FIG. 4F). Bulk RNAseq at 4 dpi confirmed that DDRR increased Top1 and Top2a (see FIG. 4I).

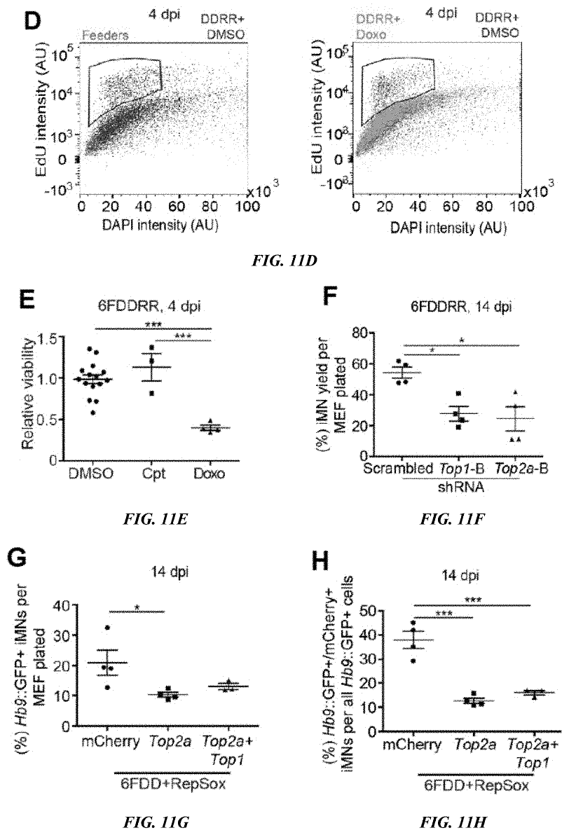

Short hairpin RNAs (shRNAs) targeting either Top1 or Top2a (see FIG. 11A) increased genomic stress and reduced HHCs in 6FDDRR conditions (see FIGS. 4J-K and FIG. 11B-C). Transient inhibition of TOP1 or TOP2A by camptothecin or doxorubicin treatment, respectively, decreased the percentage of HHCs in 6FDDRR conditions (see FIG. 4L). Doxorubicin reduced active DNA synthesis to levels of irradiated MEFs (see FIG. 11D) and only impacted cell viability modestly relative to the drop in HHCs and proliferating cells (see FIG. 4L, and FIGS. 11D-E). Consistent with HHCs comprising most of the reprogramming-competent cells, shRNA knockdown or chemical inhibition of either TOP1 or TOP2A resulted in a significant drop in iMN yield with 6FDDRR (see FIGS. 4M-N, and FIG. 11F). Although overexpression of Top2a did not increase conversion (see FIGS. 11G-H), Top2a is only transiently induced during 6FDDRR reprogramming (see FIG. 4F), and constitutive overexpression may prohibit IMN formation. Top1 overexpression significantly increased iMN conversion (see FIG. 4O, and FIG. 11I). Thus, DDRR upregulates topoisomerases to promote HHC formation and enable highly efficient reprogramming.

DDRR and Topoisomerases Reduce Negative DNA Supercoiling and R-Loop Formation and Sustain Transcription in S Phase. Transcription and DNA replication increase positive and negative supercoiling in the genome. Negative supercoiling promotes R-loop formation, which in turn can stall DNA replication forks. To investigate whether reprogramming perturbed supercoiling, cells were incubated with trimethylpsoralen (TMP), which preferentially intercalates into negatively supercoiled DNA (i.e., underwound) and signifies the amount of negative supercoiling in the genome. Because DNA synthesis can influence DNA supercoiling, DNA synthesis was normalized in Control-Pure, 6F, and 6FDDRR conditions before detecting supercoiling by using aphidicolin to inhibit DNA polymerases.

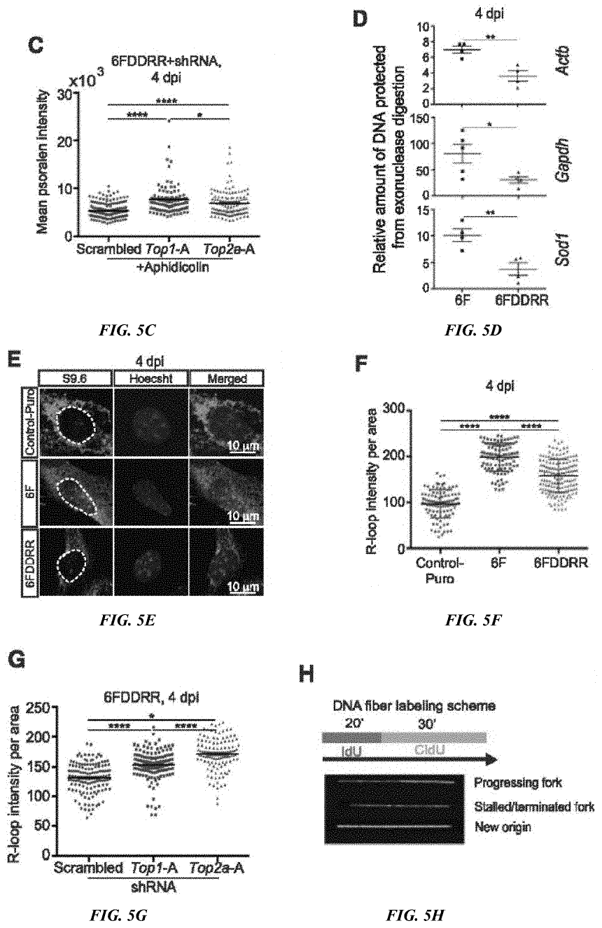

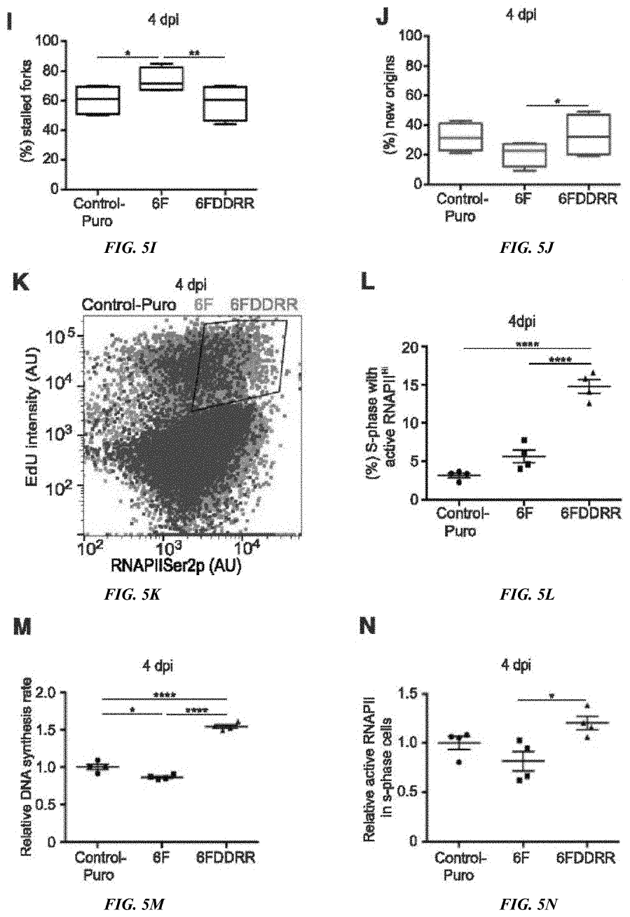

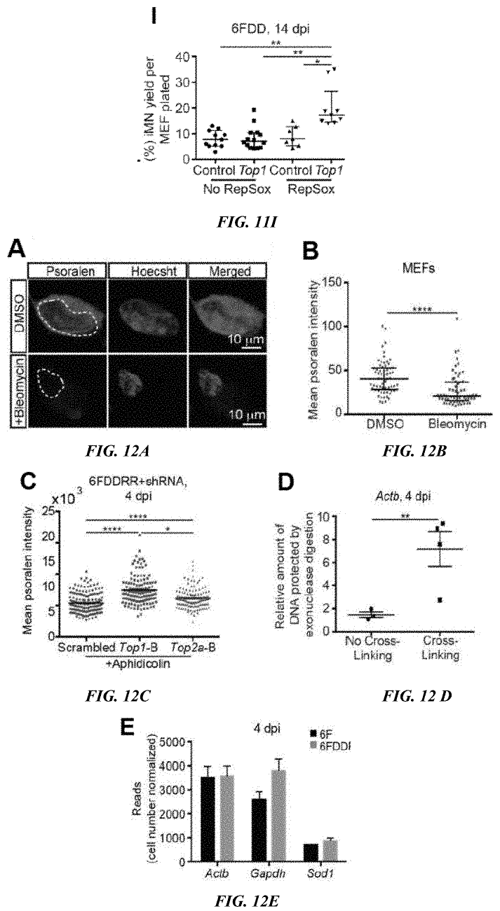

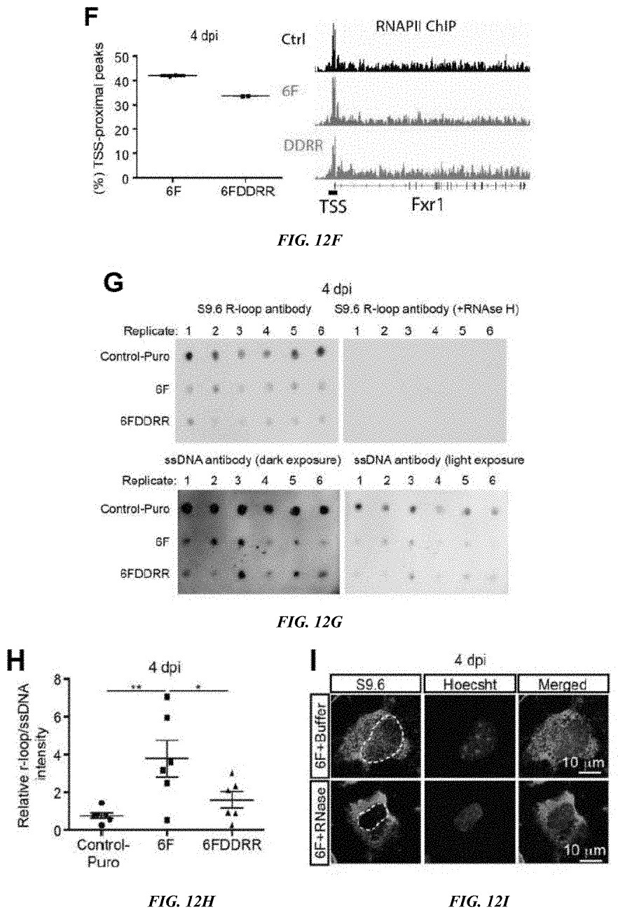

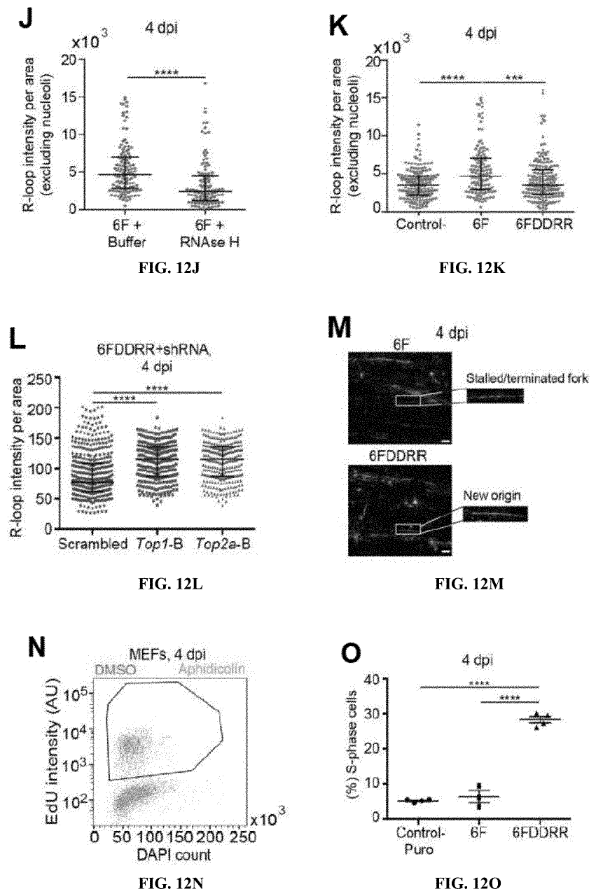

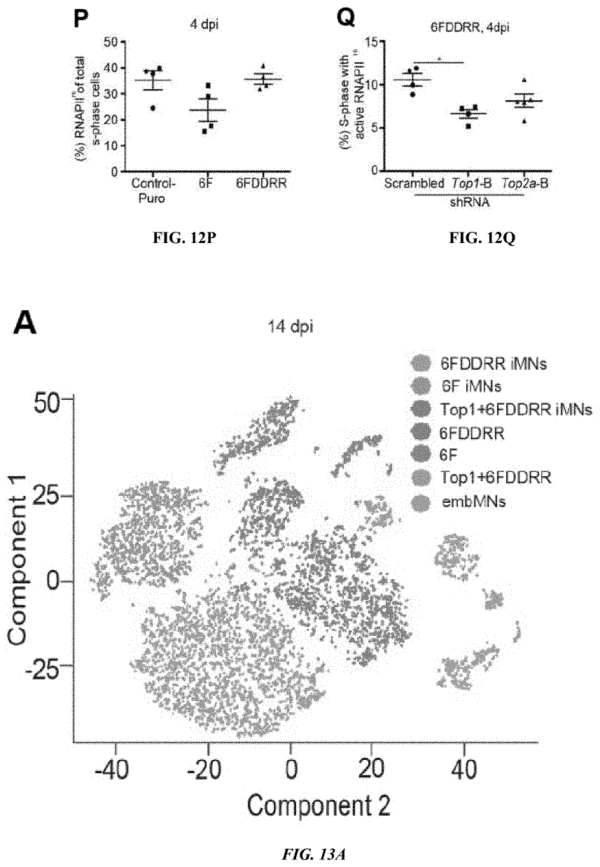

Bleomycin treatment, which causes DNA double-strand breaks and decreases DNA supercoiling, decreased biotinylated TMP intercalation (see FIG. 12A-B). At 4 dpi, 6F MEFs showed increased biotinylated TMP intercalation compared to Control-Pure MEFs, indicating that transcription factor overexpression increased negative supercoiling (see FIG. 5A-B). Addition of DDRR to 6F normalized TMP incorporation (see FIGS. 5A-B), and shRNA knockdown of Top1 or Top2a in 6FDDRA cells blocked this reduction (see FIG. 5C, and FIG. 12C).

Transcription bubbles induce negative supercoiling upstream of the transcription start site (TSS). TMP cross-linking protects negatively supercoiled genomic regions against digestion with exonuclease I and enables their quantification by qPCR. Indeed, in Control-Puro-infected cells at 4 dpi, the promoter region upstream of the Actb transcription start site was significantly more protected from exonuclease I digestion with TMP cross-linking than without (see FIG. 12D). At 4 dpi, 6FDDRA cells had significantly less negative supercoiling than 6F cells at the promoters of three genes with similar expression in 6F and 6FDDRA cells (Gapdh, Actb, and Sod1; FIG. 12E and FIG. 5D). Chromatin immunoprecipitation sequencing (ChiP-seq) showed that RNAPII bound a greater fraction of TSS-proximal peaks in 6F compared to 6FDDRA (see FIG. 12F), suggesting that DDAR reduces ANAPII pausing. Thus, 6F transduction increases negative DNA supercoiling and DDRR reduces this in a topoisomerase-dependent manner.

Negatively supercoiled DNA and high transcription rates promote A-loops, hybrid structures formed between genomic DNA, and nascent transcripts that can impair DNA replication. Using an A-loop-specific anti body (S9.6), dot blot analysis was employed (See FIGS. 12-H) and immunofluorescence (see FIGS. 5E-F, and FIGS. 12I-K) to quantify A-loops at 4 dpi in Control-Puro, 6F, and 6FDDAR conditions. As expected, RNase H reduced S9.6 signal intensity in lysates (see FIG. 12G) and non-nucleolar nuclear regions (see FIGS. 12I-J). 6F cells showed increased R-loop formation compared to Control-Pure cells (see FIGS. 5E-F, and FIGS. 12G-H and K). DDRR reduced R-loop formation compared to 6F (see FIGS. 5E-F, FIGS. 12G-H, and K), and shRNA knockdown of Top1 or Top2a increased A-loops in 6FDDAA (see FIG. 5G, and FIG. 12L).

To determine whether increased DNA supercoiling and A-loops after 6F transduction impedes DNA replication, DNA fiber labeling was used. Pulse labeling of IdU for 20 min followed by CldU for 30 min yields patterns of IdU and CldU marking progressing forks (IdU and CldU labeling), stalled forks (only IdU labeling), and new origins (only CIdU labeling; FIG. 5H). 6F increased stalled replication forks at 4 dpi, and DDAA mitigated this effect (see FIG. 5I and FIG. 12M, top). Additionally, 6FDDAA MEFs initiated more new replication origins than SF cells (see FIG. 5J and FIG. 12M).

To measure transcriptional activity in S phase cells, RNAPIISer2p levels were examined by immunolabeling and DNA synthesis by EdU (see FIG. 5K). Aphidicolin-treated cells did not incorporate EdU and provided a negative control to gate S phase cells (see FIG. 12N). 6FDDRR cultures had 3-fold higher ANAPIISer2p S phase cells than Control-Puro and 6F (see FIG. 5L). Additionally, EdU and ANAPIISer2p intensity were higher in 6FDDAA compared to 6F (see FIGS. 5M-N, and FIGS. 12O-P). Knockdown of Top1, but not Top2a, reduced the percentage of S phase cells with high ANAPIISer2p (see FIGS. 5O-P, and FIG. 12Q). Thus, 6F expression induces genomic stress by increasing negative DNA supercoiling, R-loop formation, and DNA replication fork stalling. DDRR rescues these stresses by activating topoisomerases.

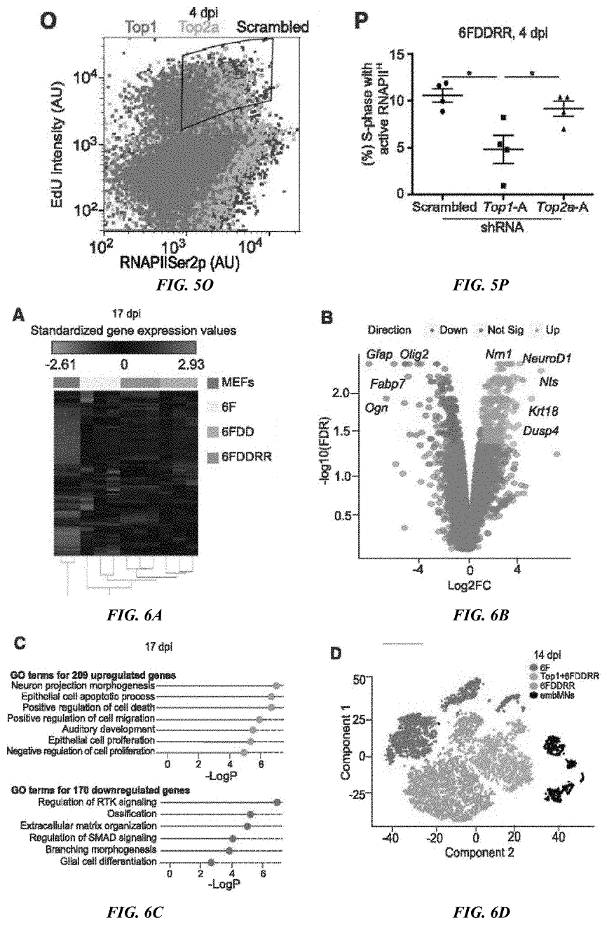

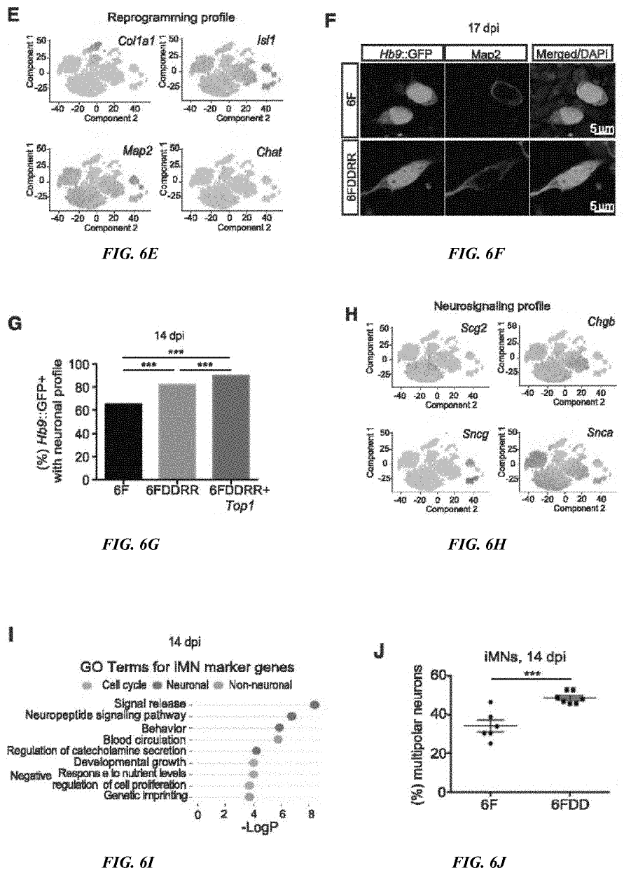

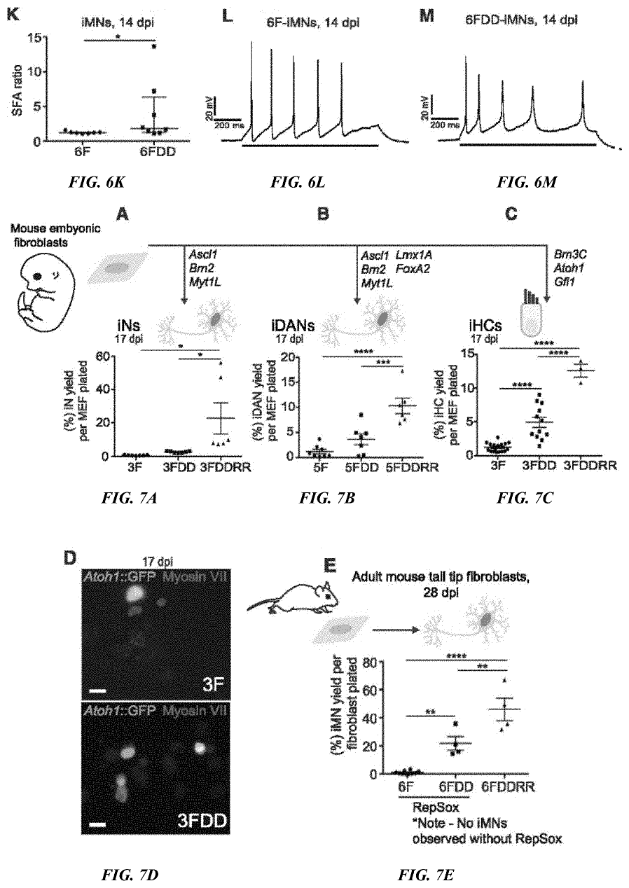

Converting HHCs Adopt the iMN Transcriptional Program, Accelerating Maturation. To determine whether DDRR affects the resulting iMNs, 6F, 6FDD, and 6FDDRR Hb9::GFP+ cells were analyzed by RNAseq. 6F and 6FDDRR Hb9::GFP+ cells were similar, although small variations differentiated them (see FIGS. 6A-C). Compared to 6F, 6FDDRRHb9::GFP+ cells downregulated fibroblast genes and upregulated genes involved in neuron projection development and processes modulated by hRasG12V, such as apoptosis, cell cycle, and migration (see FIG. 6-C). Thus, DDRR accelerates the shift toward the iMN profile generated by 6F.

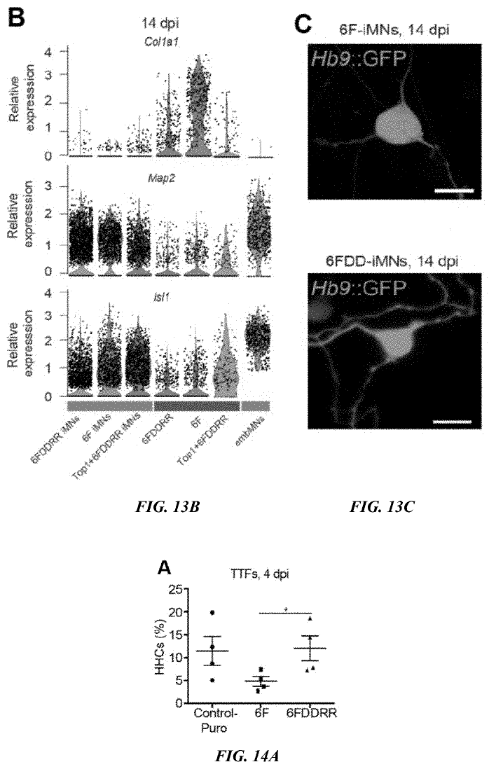

In single-cell RNA-seq analysis of primary embryonic motor neurons at embryonic day 12.5 (E12.5) and 6F, 6FDDRR, and 6FDDRR+Top1 iMNs, each iMN condition grouped into multiple clusters, each with a larger Map2+ population and a smaller Col1a1+ cluster (see FIGS. 6D-E, and FIGS. 13A-B). Immunostaining confirmed high MAP2 levels in Hb9::GFP+ cells (see FIG. 6F) and most cells grouped into the Map2+, neuronal population for each condition (see FIG. 6G). Small gene expression differences distinguished iMN clusters, including variations in neurosignaling and cell cycle (see FIGS. 6H-I).

To determine whether expanding HHCs accelerates maturation, morphological and electrophysiological properties were examined. Mature spinal motor neurons are multipolar. DD significantly increased the percentage of multipolar iMNs (see FIG. 6J and FIG. 13C). Upon repetitive stimulation, mature neurons display spike-frequency adaptation (SFA), increasing the time interval between spikes. Unlike 6F iMNs, several 6FDD iMNs achieved SFA, with an SFA ratio several-fold higher than 6F iMNs (see FIGS. 6K-M) and iPSC motor neurons with prolonged culture. Thus, expanding HHCs improves maturation.

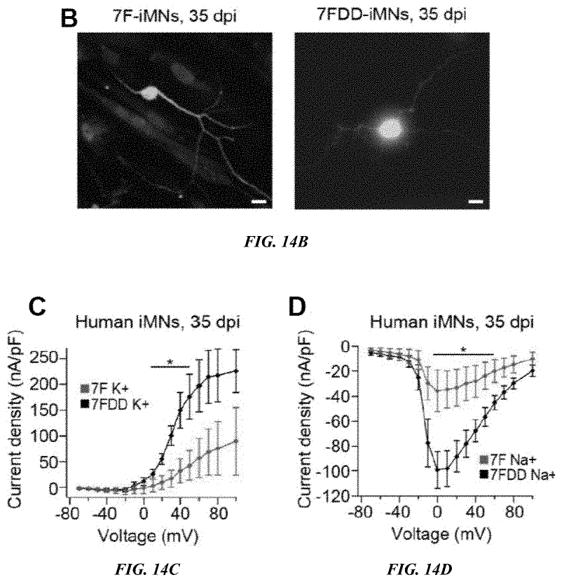

Chemical and Genetic Factors that Increase HHCs Promote Reprogramming across Cell Types and Species. Next was assessed the generality of inducing this HHC population in other reprogramming schemes. DD or DDRR increased reprogramming of MEFs into induced neurons via Ascl1, Bm2, and Mytl1L; induced dopaminergic neurons QDANs) via Ascl1, Bm2, Mytl1L, Lmx1A, and FoxA2; and induced hair cells (iHCs) via Atoh1, Gata3, and Bm3C (see FIG. 7A-D). The reprogramming increase into iMNs extended across age and species in the starting cells to include mouse adult tail tip fibroblasts and myoblasts (see FIGS. 7E-F) and human adult fibroblasts (see FIG. 7G). Although human fibroblasts reprogrammed less efficiently than mouse fibroblasts (compare FIG. 7E versus FIG. 7G), the rates of HHC and iMN formation in the 6FDDRR condition were similar between mouse embryonic and adult fibroblasts (see FIG. 2M versus FIG. 14A, and FIG. 1L versus FIG. 7E). DD increased the percentage of multipolar human iMNs (see FIG. 7H and FIG. 14B) and resulted in faster sodium and potassium currents (see FIGS. 7I-J, and FIGS. 14C-D) and tighter, more mature action potentials (see FIGS. 7K-L). Thus, HHCs promote reprogramming into post-mitotic lineages across age and species.

Numerous modifications and variations in the invention as set forth in the above illustrative examples are expected to occur to those skilled in the art. Consequently, only such limitations as appear in the appended claims should be placed on the invention.

What is claimed:

1. A method of producing a population of hypertranscribing, hyperproliferating cells (HHCs), comprising
contacting a population of cells with a cocktail that comprises a TGF-β inhibitor, and a dominant negative p53 mutant, to form a population of HHCs; and
isolating the population of HHCs.

2. The method of claim 1, wherein the TGF-β inhibitor is selected from RepSox, SB431542, A-83-01, LY-364947, LY2157299, LY-364947, A 83-01, and ALK5 inhibitor.

3. The method of claim 2, wherein the TGF-β inhibitor is RepSox.

4. The method of claim 1, wherein the dominant negative p53 mutant lacks a DNA-binding domain.

5. The method of claim 4, wherein the dominant negative p53 mutant is p53DD.

6. The method of claim 1, wherein the cocktail further comprises a Ras mutant.

7. The method of claim 6, wherein the Ras mutant is hRAS G12V.

8. The method of claim 1, wherein the cocktail relieves DNA supercoiling by activating topoisomerases.

9. The method of claim 1, wherein the cells are somatic cells.

10. The method of claim 1, wherein the cells are stem cells.

11. The method of claim 10, wherein the stem cells are embryonic stem cells or induced stem cells.

12. The method of claim 1, wherein the cells are induced motor neuronal cells (iMNs).

13. The method of claim 12, wherein the iMNs are derived from fibroblasts.

14. The method of claim 1, wherein the population of HHCs is converted into neurons.

15. The method of claim 13, wherein the neurons are characterized as being electrophysiology mature.

16. A method of producing induced pluripotent stem cell, comprising:
contacting a somatic cell with a cocktail that comprises a TGF-β inhibitor, and a dominant negative p53 mutant, to form a population of HHCs;
isolating the population of HHCs;
contacting the HHCs with at least one dedifferentiation factor to under conditions to produce induced pluripotent stem cells from the HHCs.

17. The method of claim 19, wherein the TGF-β inhibitor is selected from RepSox, SB431542, A-83-01, LY-364947, LY2157299, LY-364947, A 83-01, and ALK5 inhibitor.

18. The method of claim 16, wherein the dominant negative p53 mutant lacks a DNA-binding domain.

19. The method of claim 16, wherein the cocktail further comprises a Ras mutant.

20. The method of claim 16, wherein the somatic cell is selected from the group consisting of a neuronal cells, a fibroblast, a hepatic cells, a pancreatic cell, a skin cells and a muscle cell.
